SEQUENCE LISTING (22 sequences)

SEQ ID NO: 1 - protein, 302 aa, artificial
  construct without RSS and GFP
MGSSKSKPKDPSQRLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGSIAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKKALLDGKASNPEQTAEEEQETATWT

SEQ ID NO: 2 - protein, 558 aa, artificial
  construct with RSS and GFP
MGSSKSKPKDPSQRLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGSIAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKKALLDGKASNPEQTAEEEQETATWTEFEGSAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAF

SEQ ID NO: 3 - DNA, 2241 bp, artificial
  construct
  EF1_promoter (1)..(558)
  Kozak_Sequence (559)..(564)
  myristoylation_domain (565)..(606)
  F36V_domain (612)..(933)
  CTLR4_sequence (939)..(1470)
  T2A_sequence (1494)..(1548)
  copGFP (1577)..(2241)
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagctagcgccaccatggggagtagcaagagcaagcctaaggaccccagccagcgcctcgagggcgtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggatccattgctggctgtaaaaagtacagcagaggagaaagcatctatgatgcatttgtgatctactcgagtcagaatgaggactgggtgagaaatgagctagtaaagaatttagaagaaggagtgccccgctttcacctctgccttcactacagagactttattcctggtgtagccattgctgccaacatcatccaggaaggcttccacaagagccggaaggttattgtggtagtgtctagacactttattcagagccgttggtgtatctttgaatatgagattgctcaaacatggcagtttctgagcagccgctctggcatcatcttcattgtccttgagaaggttgagaagtccctgctgaggcagcaggtggaattgtatcgccttcttagcagaaacacctacctggaatgggaggacaatcctctggggaggcacatcttctggagaagacttaaaaaggccctattggatggaaaagcctcgaatcctgagcaaacagcagaggaagaacaagaaacggcaacttggaccgaattcgaaggatccgcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgcc

SEQ ID NO: 4 - protein, 559 aa, artificial
  construct protein sequence
  myristoylation_domain (1)..(15)
  F36V_domain (16)..(123)
  cTLR4_domain (130)..(302)
  T2A_sequence (310)..(328)
  copGFP (338)..(559)
  copGFP (343)..(559)
MGSSKSKPKDPSQRLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGSIAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKKALLDGKASNPEQTAEEEQETATWTEFEGSAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFA

SEQ ID NO: 5 - DNA, 1677 bp, Mus musculus
atggggagtagcaagagcaagcctaaggaccccagccagcgcctcgagggcgtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggatccattgctggctgtaaaaagtacagcagaggagaaagcatctatgatgcatttgtgatctactcgagtcagaatgaggactgggtgagaaatgagctagtaaagaatttagaagaaggagtgccccgctttcacctctgccttcactacagagactttattcctggtgtagccattgctgccaacatcatccaggaaggcttccacaagagccggaaggttattgtggtagtgtctagacactttattcagagccgttggtgtatctttgaatatgagattgctcaaacatggcagtttctgagcagccgctctggcatcatcttcattgtccttgagaaggttgagaagtccctgctgaggcagcaggtggaattgtatcgccttcttagcagaaacacctacctggaatgggaggacaatcctctggggaggcacatcttctggagaagacttaaaaaggccctattggatggaaaagcctcgaatcctgagcaaacagcagaggaagaacaagaaacggcaacttggaccgaattcgaaggatccgcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgcc

SEQ ID NO: 6 - protein, 179 aa, Homo sapiens
LAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 7 - protein, 179 aa, homo sapiens
LAGCIKYGRGENIYDAFVIYSSQDEDWVRNELVKNLEEGVPPFQLCLHYRDFIAGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 8 - protein, 179 aa, homo sapiens
LAGCIKYGRGENIYDAFVIYSSQDEDWVRNELVKNLEEGVPPFQLCLHYRDFIPGVAIAANIIHEGFHKSRKVIVVVSQHFIQARWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 9 - protein, 179 aa, homo sapiens
LAGCIKYGRGENIYDAFVIYSSQDEDWVRNELVKNLEEGVPPFQLCLHYRDFIPGVAIAANIIHEGFHKSRKVIVVVSQHFIQSAWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 10 - protein, 179 aa, homo sapiens
LAGCIKYGRGENIYDAFVIYSSQDEDWVRNELVKNLEEGVPPFQLCLHYRDFIPGVAIAANIIHEGFHKSRKVIVVVSQHFIQSRWSIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 11 - protein, 179 aa, homo sapiens
LAGCIKYGRGENIYDAFVIYSSQDEDWVRNELVKNLEEGVPPFQLCLHYRDFIPGVAIAANIIHEGFHKSRKVIVVVSQHFIQSRWCIFEAEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 12 - protein, 179 aa, homo sapiens
LAGCIKYGRGENIYDAFVIYSSQDEDWVRNELVKNLEEGVPPFQLCLHYRDFIPGVAIAANIIHEGFHKSRKVIVVVSQHFIQSRWCIFEYAIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 13 - protein, 179 aa, homo sapiens
LAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVAKTLLRQQVELYRLLSRNTYLEWEDSVLGRHIFWRRLRKALLDGKSWNPEGTVGTGCNWQEATSI

SEQ ID NO: 14 - protein, 179 aa, homo sapiens
LAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVESTLLRQQVELYRLLSRNTYLEWEDSVLGRHIFWRRLRKALLDGKSWNPEGTVGTGCNWQEATSI

SEQ ID NO: 15 - protein, 179 aa, homo sapiens
LAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRAQVELYRLLSRNTYLEWEDSVLGRHIFWRRLRKALLDGKSWNPEGTVGTGCNWQEATSI

SEQ ID NO: 16 - protein, 179 aa, Homo sapiens
LAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRATYLEWEDSVLGRHIFWRRLRKALLDGKSWNPEGTVGTGCNWQEATSI

SEQ ID NO: 17 - protein, 179 aa, Homo sapiens
LAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTALEWEDSVLGRHIFWRRLRKALLDGKSWNPEGTVGTGCNWQEATSI

SEQ ID NO: 18 - protein, 179 aa, Homo sapiens
LAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLAWEDSVLGRHIFWRRLRKALLDGKSWNPEGTVGTGCNWQEATSI

SEQ ID NO: 19 - protein, 179 aa, Homo sapiens
LAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWADSVLGRHIFWRRLRKALLDGKSWNPEGTVGTGCNWQEATSI

SEQ ID NO: 20 - protein, 179 aa, Homo sapiens
LAGCIKYGRGENIYDAFVIYSSQDEDWVRNELVKNLEEGVPPFQLCLHYRDFIAGVAIAANIIHEGFHKSRKVIVVVSQHFIQAAWSIFEAAIAQTWQFLSSRAGIIFIVLQKVASTLLRAQVELYRLLSRATALAWADSVLGRHIFWRRLRKALLDGKSWNPEGTVGTGCNWQEATSI

SEQ ID NO: 21 - protein, 835 aa, Homo sapiens
MMPPWLLARTLIMALFFSCLTPGSLNPCIEVVPNITYQCMDQKLSKVPDDIPSSTKNIDLSFNPLKILKSYSFSNFSELQWLDLSRCEIETIEDKAWHGLHHLSNLILTGNPIQSFSPGSFSGLTSLENLVAVETKLASLESFPIGQLITLKKLNVAHNFIHSCKLPAYFSNLTNLVHVDLSYNYIQTITVNDLQFLRENPQVNLSLDISLNPIDFIQDQAFQGIKLHELTLRGNFNSSNIMKTCLQNLAGLHVHRLILGEFKDERNLEIFEPSIMEGLCDVTIDEFRLTHTNDFSDDIVKFHCLANVSAMSLAGVSIKYLEDVPKHFKWQSLSIIRCQLKQFPTLDLPFLKSLTLTMNKGSISFKKVALPSLSYLDLSRNALSFSGCCSYSDLGTNSLRHLDLSFNGAIIMSANFMGLEELQHLDFQHSTLKRVTEFSAFLSLEKLLYLDISYTNTKIDFDGIFLGLTSLNTLKMAGNSFKDNTLSNVFANTTNLTFLDLSKCQLEQISWGVFDTLHRLQLLNMSHNNLLFLDSSHYNQLYSLSTLDCSFNRIETSKGILQHFPKSLAFFNLTNNSVACICEHQKFLQWVKDQKQFLVNVEQMTCATPVEMNTSLVLDFNNSTCYMYKTIISVSVISVIVVSTVAFLIYHFYFHLILIAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKKALLDGKASNPEQTAEEEQETATWT

SEQ ID NO: 22 - DNA, 531 bp, Homo sapiens
  Xho_1 (60)..(65)
attgctggctgtaaaaagtacagcagaggagaaagcatctatgatgcatttgtgatctactcgagtcagaatgaggactgggtgagaaatgagctggtaaagaatttagaagaaggagtgccccgctttcacctctgccttcactacagagactttattcctggtgtagccattgctgccaacatcatccaggaaggcttccacaagagccggaaggttattgtggtagtgtctagacactttattcagagccgttggtgtatctttgaatatgagattgctcaaacatggcagtttctgagcagccgctctggcatcatcttcattgtccttgagaaggttgagaagtccctgctgaggcagcaggtggaattgtatcgccttcttagcagaaacacctacctggaatgggaggacaatcctctggggaggcacatcttctggagaagacttaaaaatgccctattggatggaaaagcctcgaatcctgagcaaacagcagaggaagaacaagaaacggcaacttggacc

(19) United States
(12) Patent Application Publication
(10) Pub. No.: US 2019/0008897 A1
(43) Pub. Date: Jan. 10, 2019

(54) COMPOSITIONS AND METHODS FOR PRODUCING PRO-INFLAMMATORY MACROPHAGES

(71) Applicant: UNIVERSITY OF WASHINGTON, SEATTLE, WA (US)

(72) Inventors: Marta SCATENA, SEATTLE, WA (US); Karen EATON, SEATTLE, WA (US); Cecilia M. GIACHELLI, SEATTLE, WA (US)

(73) Assignee: UNIVERSITY OF WASHINGTON, SEATTLE, WA (US)

(21) Appl. No.: 15/746,780

(22) PCT Filed: Jul. 22, 2016
(86) PCT No.: PCT/US16/43540

§ 371 Date: Jan. 22, 2018

Related U.S. Application Data
(60) Provisional application No. 62/195,725, filed on Jul. 22, 2015.

(51) Int. Cl. A61K 35/15; C07K 14/705; C07K 14/435; A61K 31/436; A61K 9/00; A61P 29/00

(52) U.S. Cl. CPC A61K 35/15; C07K 14/70596; C07K 14/43595; A61K 48/00; A61K 9/0019; A61P 29/00; A61K 31/436

(57) ABSTRACT

Constructs are provided that exploit selected portions of the TLR4 receptor to which no extracellular ligand will bind. A dimerization domain is employed to allow for regulated activation when used in conjunction with a dimerizer drug. In addition, a myristoylation domain facilitates intracellular presentation. Constructs of the invention can be used to create engineered monocytes whose TLR4 receptors can be selectively activated with administration of a dimerization agent. Engineered macrophages can be selectively induced to become pro-inflammatory, providing methods to ameliorate conditions associated with excessive pro-repair macrophages, such as cardiac fibrosis and solid tumor growth. Delivery of the engineered macrophages to sites of cardiac fibrosis can reduce the amount of fibrosis and scarring and ameliorate cardiac function. Delivery of the engineered macrophages to solid tumors can reduce tumor growth and size.

This application claims benefit of U.S. provisional patent application No. 62/195,725, filed Jul. 22, 2015, the entire contents of which are incorporated by reference into this application.

### REFERENCE TO A SEQUENCE LISTING SUBMITTED VIA EFS-WEB

The content of the ASCII text file of the sequence listing named “UW61WOU1_SL”, which is 50 kb in size, was created on Jul. 20, 2016, and electronically submitted via EFS-Web herewith the application. The sequence listing is incorporated herein by reference in its entirety.

### TECHNICAL FIELD OF THE INVENTION

This invention relates to methods, constructs and compositions that can be used to produce pro-inflammatory macrophages. Compositions comprising engineered monocytes can be administered to a subject in need of treatment with pro-inflammatory macrophages. The polarization of the engineered monocytes can be regulated using a ligand, allowing for both spatial and temporal control of the treatment.

### BACKGROUND OF THE INVENTION

Macrophages, a main inflammatory cell type, are known to be key players in the inflammatory response. When activated, they exist in two major phenotypes that can be broadly defined as: pro-inflammatory and pro-repair macrophages. Pro-inflammatory macrophages are the first to arise at the site of injury and propagate the initial response by releasing pro-inflammatory cytokines as well as by producing reactive oxygen species in order to destroy foreign material at the injury site (1). Pro-repair macrophages promote growth and regeneration and are present following the pro-inflammatory macrophage decline. These types of macrophages are present toward the end of the inflammatory response and mainly function to end and resolve inflammation, stimulate healing, and restore tissue homeostasis that is characterized by proper vascularization and little to no fibrosis or scarring (2).

In most inflammation scenarios, having a local overabundance of pro-inflammatory macrophages can potentially lead to chronic inflammatory diseases, like atherosclerosis and rheumatoid arthritis. Conversely, having a local overabundance of pro-repair macrophages can lead to fibrotic diseases, like pulmonary and cardiac fibrosis. Pro-repair macrophages are also found associated with solid tumors. Tumors cells are thought to induce the pro-repair macrophage phenotype that in turn allows for tumor progression and tumor vascularization (1).

Experimental evidence suggests that pro-inflammatory and pro-repair macrophages have the ability to regulate one another and impact the state of inflammation, most likely depending on the expression of the cytokines present in the local environment. These notions strengthen the theory that it is necessary to have a balance of pro-inflammatory macrophages and pro-repair macrophages and any skewing of this balance could potentially lead to the dysregulation of inflammation and associated diseases (1-2).

There remains a need for effective means of selectively inducing and regulating pro-inflammatory macrophages.

### SUMMARY OF THE INVENTION

The invention meets these needs and others by providing methods and compositions that mediate selective inducement of pro-inflammatory macrophages. The invention provides, in one embodiment, a polynucleotide construct encoding a chimeric cellular receptor. In one embodiment, the construct comprises nucleic acid sequences encoding, in operable linkage, a myristoylation sequence (Myr), a dimerizer domain, and an intracellular portion of the TLR4 receptor. In some embodiments, the construct further comprises a sequence that enables expression of a separate reporter molecule controlled by the same promoter. Examples of such a sequence include, but are not limited to, a ribosome skipping sequence and a cleavage sequence. One example of a reporter molecule is green fluorescent protein (GFP). In one embodiment, the construct is free of sequences encoding an extracellular portion of the TLR4 receptor. In preferred embodiments, the construct is sufficiently free of sequences encoding an extracellular portion of the TLR4 receptor that no extracellular ligands will bind. In one embodiment, the dimerizer domain is phenylalanine 36 to valine point mutation (F36V) derived from a mutated version of the endogenous FKBP12 protein.

In one embodiment, the nucleic acid sequences encoding the myristoylation sequence (Myr), the dimerizer domain, and the intracellular portion of the TLR4 receptor comprises a nucleotide sequence encoding an amino acid sequence having at least 90% sequence identity to the amino acid sequence of SEQ ID NO: 1. In another embodiment, the nucleic acid sequences encoding the myristoylation sequence (Myr), the dimerizer domain, the intracellular portion of the TLR4 receptor, the ribosome skipping sequence, and the green fluorescent protein comprises a nucleotide sequence encoding an amino acid sequence having at least 90% sequence identity to the amino acid sequence of SEQ ID NO: 2.

In one embodiment, the intracellular portion of the TLR4 receptor comprises an amino acid sequence having at least 90% sequence identity to the amino acid sequence of LAGCIKYGRGENIYDAFVIYSSQDEDWVRNELVKNLEEGVPPFQLCLHYRDFIPGVAIAA NIIHEGFHKSRKVIVVVSQHFIQSRWCIFEYEIAQTWQFLSSRAGIIFIVLQKVEKTLLRQQ VELYRLLSRNTYLEWEDSVLGRHIFWRRLRKALLDGKSWNPEGTVGTGCNWQEATSI (SEQ ID NO: 6), In some embodiments, the intracellular portion of the TLR4 receptor includes one or more of the following mutations: P714A, S744A, R745A, C747S, Y751A, E752A, E775A, K776S, Q792A, N792 A, Y794A, E796A, and E798A, wherein amino acid numbering refers to the human TLR4 receptor protein, amino acid residues 661-839 of which are shown in SEQ ID NO: 6. Representative examples of the intracellular portion of the TLR4 receptor include, but are not limited to, polypeptides comprising an amino acid sequence selected from those shown in SEQ ID NO: 7-19, or SEQ ID NO: 20, the latter of which includes each of the point mutations indicated above.

The invention further provides a method of producing a genetically engineered monocyte. In one embodiment_{;} the method comprises the steps of: (a) contacting a monocyte with a construct as described herein under conditions sufficient to transfect the construct into the monocyte; and (b) culturing the monocyte transfected in step (a). The genetically engineered monocytes express the chimeric cellular receptor able to dimerize upon addition of a Chemical Inducer of Dimerization (CID) synthetic ligand, which dimerization activates signaling pathways independently of endogenous physiological ligands, thereby differentiating the transfected monocyte into a macrophage. In one embodiment, the CID synthetic ligand is a recombinant FK506 molecule. In one embodiment, the recombinant FK506 molecule is AP20187.

Also provided is a genetically engineered monocyte produced in accordance with the methods described herein, as well as a pharmaceutical composition comprising a genetically engineered monocyte of the invention. In some embodiments, the composition further comprises a CID synthetic ligand. In one embodiment, the CID synthetic ligand is a recombinant FK506 molecule, such as, for example, AP20187.

The invention additionally provides a method of reversibly inducing pro-inflammatory macrophages in a subject. In one embodiment, the method comprises: (a) administering the composition of claim 11 to a subject; and (b) administering a CID synthetic ligand to the subject. The CID synthetic ligand activates the genetically engineered monocytes. In one embodiment, the method further comprises: (c) withdrawing administration of the CID synthetic ligand and/or administering a washout ligand, thereby reversing the activation of the genetically engineered monocytes. The administering of step (a) can be via means suitable to the particular patient and treatment objective, such as by implantation into a target organ, injection into a target tissue, introduction of a scaffold to a target site, or intravenous administration. In some embodiments, the genetically engineered monocyte is pre-treated with a CID synthetic ligand prior to the administering of step (a). In such embodiments, pre-polarized cells are administered to the subject, thereby jump-starting the process. The CID synthetic ligand is administered subsequently to maintain the cells in a polarized state in vivo.

The invention provides methods of treating conditions that would benefit from selective inducement of pro-inflammatory macrophages. In one embodiment, the invention provides a method of treating a fibrotic disease comprising administering to a subject in need thereof the pharmaceutical composition of the invention and a CID synthetic ligand. Representative examples of fibrotic disease include, but are not limited to, those selected from the group consisting of pulmonary fibrosis, cardiac fibrosis, and foreign body reaction. In another embodiment, the invention provides a method of treating an inflammatory disease comprising administering to a subject in need thereof the pharmaceutical composition of the invention and a CID synthetic ligand. In one embodiment, the inflammatory disease is a chronic inflammatory disease. Representative examples of chronic inflammatory disease include, but are not limited to, those selected from the group consisting of atherosclerosis and rheumatoid arthritis. In yet another embodiment, the invention provides a method of treating cancer inflammation comprising administering to a subject in need thereof the pharmaceutical composition of the invention and a CID synthetic ligand.

### BRIEF DESCRIPTION OF THE DRAWINGS

FIG. 1. Engineered MΦs utilizing the Chemically Induced Dimerization (CID) system. Schematic representation of the engineered macrophages and the activation by CID drug.

FIG. 2. Diagram of cTLR4 Construct and Engineered Receptor and Confirmation of cTLR4 construct expression. (A) The cTLR4 construct contains: a myristolation domain (Myr), an engineered dimerization domain (F36V), the cytoplasmic portion of the TLR4 domain (cTLR4), a T2A ribosome skipping sequence, and a GFP tag. (B) Western blot probing for the FKBP12/F36V domain (35.5 kDa) containing lysates from RAW264.7 cells, MΦ-T2A negative control cells, MΦ-cTLR4 cells, and F2 positive control cells. MΦ-T2A cells have been transduced with a construct that contains only the T2A ribosome skipping sequence with the GFP tag. F2 cells have been transduced with just two adjacent F36V domains. Schematic of the cTLR4 DNA construct inserted. Myr=myristolation sequence, F36V=dimerizer domain, cTLR4=intracellular portion of the TLR4 receptor, T2A=cleavage sequence, GFP=Green Fluorescent Protein.

FIG. 3. Mouse F36V-cTLR4 DNA sequence (SEQ ID NO: 5).

FIG. 4. Mouse F36V-cTLR4 Amino Acid sequence (SEQ ID NO: 4).

FIG. 5. Residues 661 to 839 human TLR4 cytoplasmic/intracellular portion of the receptor (SEQ ID NO: 6). Mutations in all three possible binding sites abrogate TLR4 TIR-TLR4 TIR interactions. Only potential binding sites I and II in the TLR4 TIR domain are critical for LPS-induced NF-κB signaling. An IRF3/GAL4-based assay suggests that binding site III may be important for IRF-3 activation.

FIG. 6. Proposed mutations in human TLR4 cytoplasmic/intracellular portion amino acid residues 661 to 839 include: P714A, S744A, R745A, C747S, Y751A, E752A, E775A, K776S, Q792A, N792 A, Y794A, E796A, and E798A (SEQ ID NO: 20).

FIG. 7. Control macrophages transduced with the pCDH-EFI-MCS-T2A-copGFP vector were also generated (T2A RAW264.7). The presence of the construct was confirmed for the MΦ-cTLR4 engineered cells via western blot analysis for the FKBP12/F36V domain with a band visible at about 35.5 kDa.

FIG. 8. CID-treated MΦ-cTLR4 Cells Exhibit Increased Expression of TNFα, IL-6, and iNOS. Expression of classical inflammatory MΦ phenotype markers, determined by sandwich ELISA assay. Bar graphs show the levels of (A) TNFα and (B) IL-6 of CID-treated (50 nM) MΦ-cTLR4 cells compared to untreated, vehicle (100% EtOH), and LPS-treated cells (100 ng/mL), Cell media was collected following 24 h treatment. (C) Western blot shows intensity of iNOS expression (130 kDa) for CID and LPS-treated MΦ-cTLR4 cells when compared to controls. Cells were lysed following 24 h treatment.

FIG. 9. MΦ-cTLR4 Cells are Influenced by IL-4 Treatment. Expression of classical inflammatory MΦ phenotype markers following cocktail treatment of IL-4 (60 ng/mL) and CID drug (50 nM), LPS (100 ng/mL) or vehicle. Bar graphs show the levels of (A) TNFα and (B) IL-6 of cocktail-treated cells compared to controls. Cell medium was collected following 24 hour treatment. (C) Western blot shows intensity of iNOS expression (130 kDa) for cocktail-treated MΦ-cTLR4 cells when compared to controls. Cells were lysed following 24 hour treatment.

FIG. 10. MΦ-cTLR4 Cells Return to Baseline Levels 48 Hours Following CID Drug Withdrawal. CID drug withdrawal experiment, determined by IL-6 expression. Cells were treated with CID drug (50 nM), LPS (100 ng/mL), or vehicle for 24 hours or left untreated for 24 hours. Media was collected at the indicated timepoints after complete CID drug withdrawal and IL-6 levels were measured at each timepoint to determine activation intensity.

FIG. 11. CID-treated MΦ-cTLR4 Cells Remain Activated for At Least 48 Hours. Longevity study determined by expression of (A) TNF-α, (B) IL-6, and (C) iNOS in MΦ-cTLR4 cells. Media containing CID drug (50 nM) was changed every 24. Cell media was collected at time points indicated.

FIG. 12. CID-treated MΦ-cTLR4 Cells Activate the MyD88-dependent Pathway. (A) Western blot probing for p-ERK and total ERK. Cells were lysed at the time points indicated following CID drug (50 nM), LPS (100 ng/mL), or vehicle (100% EtOH) treatment. Top panel shows phosphorylated over total ERK ratio relative to the zero timepoint for each subsequent timepoint. Bottom panel shows corresponding blots. (B) A Dual-luciferase® assay was used to determine NF-κB activity in MΦ-cTLR4 cells. Luciferase activity was measured 4 hours following treatment of MΦ-TLR4 cells with CID drug or vehicle treatment. Luciferase activity was normalized to baseline Renilla luciferase activity.

FIG. 13. CID-treated MΦ-cTLR4 Cells Activate the MyD88-independent Pathway. Western blot for p-IRF3 and total IRF3. Cells were lysed at timepoints indicated following CID drug (50 nM), LPS (100 ng/mL), or vehicle treatment.

FIG. 14. Media From CID-treated MΦ-cTLR4 Cells Upregulate VCAM-1 and ICAM-1 on Endothelial Cells. Endothelial activation, determined by flow cytometry. Conditioned media from MΦ-cTLR4 cells treated for 6 hours with TNFα, CID, and vehicle was transferred to plated bEnd.3 endothelial cells and left to incubate for 12 hours. Following the 12 hour incubation, bEnd.3 cells were then trypsinized and stained for both VCAM-1 and ICAM-1. (A&B) Histograms showing intensity of VCAM-1 and ICAM-1 expression on bEnd.3 cells incubated with MΦ-TLR4 conditioned media treated with TNFα (20 ng/mL), CID (50 nM), or vehicle (C&D) Histograms showing intensity of VCAM-1 and ICAM-1 expression on bEnd.3 cells incubated with MΦ-TLR4 conditioned media treated with TNFα (20 ng/mL), CID (50nM), or vehicle, as well as with or without TNFα neutralizing antibody (α-TNFα).

FIG. 15. Dose of 50 nM CID Drug Produces Maximum Activation of MΦ-cTLR4 Cells. CID drug dosage optimization for MΦ-cTLR4 cells, determined by IL-6 expression. An IL-6 ELISA was performed to test for the maximum signal of this cytokine in a CID drug titration experiment. CID drug doses ranged from 50 nM to 250 nM and CID-treated MΦ-cTLR4 cells were compared to controls. Cells media was collected following 24 hour treatment.

FIG. 16. MidLow MΦ-cTLR4 Cells Exhibit Highest Signal to Noise Ratio. An IL-6 ELISA was performed on unsorted cells and four groups of sorted cells from the lowest to the most intense GFP intensity of MΦ-cTLR4 cells. Cells were treated with CID drug (50 nM), LPS (100 ng/mL), or vehicle for 24 hours or left untreated for 24 hours.

FIG. 17. MΦ-cTLR4 Cells are influenced by IL-4 Treatment. Expression of the IL-10 cytokine (M2 marker) following cocktail treatment of IL-4 (60 ng/mL) and CID drug (50 nM), LPS (100 ng/mL), or vehicle. Bar graph shows the levels of IL-10 for cocktail-treated cells compared to controls. Cell medium was collected following 48 hour treatment.

FIG. 18. Co-culture Angiogenesis Assay Results. Top left panel shows example of the angiogenesis analyzer, which uses different colors to distinguish branches, twigs, segments, master segments, meshes, nodes surrounded by junctions, master junctions, isolated elements, small isolated elements, and extremities. Bottom left panel shows network formation for vehicle-treated MΦ-cTLR4 cells co-cultured with RAECs. Top right panel shows network formation for CID-treated (50 nM) MΦ-cTLR4 cells co-cultured with RAECs. Bottom right panel shows network formation for LPS-treated (100 ng/mL) MΦ-cTLR4 cells co-cultured with RAECs.

FIG. 19. Quantification of Co-culture Angiogenesis Assay. (A) Bar graph of quantification of master segments, (B) Bar graph of quantification of isolated segments. (C) Bar graph of quantification of branch number, (D) Bar graph of quantification of branching interval.

FIG. 20. Schematic illustration showing how MΦ-cTLR4 cells were tested in vivo using a Matrigel plug. Liquid Matrigel with MΦ-cTLR4 cells were s.c. injected into a mouse and left to solidify. Mice were injected with CID drug every other day with 2 mg/kg mouse weight. Matrigel plugs were then removed following 7 or 14 days and analyzed.

FIG. 21. 14 Day CID-treated Plugs with GFP and iNOS Positive Cells with Co-localization. Image taken at 40× magnification. Top left panel shows Dapi staining of nuclei, top right panel shows GFP positive cells, bottom left panel shows iNOS positive cells, and bottom right panel shows the merged image.

FIG. 22. ELISA for VEGF in the supernatant of TLR4 engineered cells. (A) ELISA for VEGF in the supernatant using T2A=control cell line, Untreated=TLR4 engineered cells with no treatment, EtOH=TLR4 engineered cells treated with vehicle, +CID=TLR4 engineered cells treated with CID, +LPS=TLR4 engineered cells treated with LPS. (B) ELISA for VEGF in the supernatant of TLR4 engineered cells. Untreated=TLR4 engineered cells with no treatment, INF-gamma=TLR4 engineered cells treated with INF-gamma, CID+INF-gamma=TLR4 engineered cells treated with CID and INF-gamma, LPS+INF-gamma=TLR4 engineered cells treated with LPS and INF-gamma, CID=TLR4 engineered cells treated with CID.

### DETAILED DESCRIPTION OF THE INVENTION

The invention is based on the surprising and unexpected discovery of a method of engineering a TLR4 receptor that is activated only in target cells. The invention provides a construct that exploits selected portions of the TLR4 receptor to which no extracellular ligand will bind. A dimerization domain is employed to allow for regulated activation when used in conjunction with a dimerizer drug. In addition, a myristoylation domain facilitates intracellular presentation. Constructs of the invention can be used to create engineered monocytes whose TLR4 receptors can be selectively activated to induce pro-inflammatory macrophages with administration of a dimerization agent. The engineered macrophages can be used to ameliorate conditions associated with excessive pro-repair macrophages, like cardiac fibrosis and solid tumor growth. Delivery of the engineered macrophages to sites of cardiac fibrosis can reduce the amount of fibrosis and scarring, and ameliorate cardiac function. Delivery of the engineered macrophages to solid tumors can reduce tumor growth and size.

### Definitions

All scientific and technical terms used in this application have meanings commonly used in the art unless otherwise specified. As used in this application, the following words or phrases have the meanings specified,

As used herein, “intracellular portion of a TLR4 receptor” means a portion of the receptor that is sufficiently free of sequences encoding an extracellular portion of the TLR4 receptor that no extracellular ligands will bind.

As used herein, “polypeptide” includes proteins, fragments of proteins, and peptides, whether isolated from natural sources, produced by recombinant techniques or chemically synthesized. Peptides of the invention typically comprise at least about 6 amino acids.

A “mutation” is an alteration of a polynucleotide sequence, characterized either by an alteration in one or more nucleotide bases, or by an insertion of one or more nucleotides into the sequence, or by a deletion of one or more nucleotides from the sequence, or a combination of these.

As used herein, “promoter” means a region of DNA, generally upstream (5′) of a coding region, which controls at least in part the initiation and level of transcription. Reference herein to a “promoter” is to be taken in its broadest context and includes the transcriptional regulatory sequences of a classical genomic gene, including a TATA box or a non-TATA box promoter, as well as additional regulatory elements (i.e., activating sequences, enhancers and silencers) that alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene, although they may also be many kb away. Promoters may contain additional specific regulatory elements, located more distal to the start site to further enhance expression in a cell, and/or to alter the timing or inducibility of expression of a structural gene to which it is operably connected.

As used herein, “operably connected” or “operably linked” and the like is meant a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the nucleic acid sequences being linked are typically contiguous and, where necessary to join two protein coding regions, contiguous and in reading frame. “Operably linking” a promoter to a transcribable polynucleotide is meant placing the transcribable polynucleotide (e.g., protein encoding polynucleotide or other transcript) under the regulatory control of a promoter, which then controls the transcription and optionally translation of that polynucleotide.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides.

As used herein, “identical” means, with respect to amino acid sequences, that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. This type of substitution can be referred to as a conservative substitution. Preferably, a conservative substitution of any of the amino acid residues contained in a given amino acid sequence, these changes have no effect on the binding specificity or functional activity of the resulting antibody when compared to the unmodified antibody.

As used herein, “corresponding position” refers to an amino acid residue that is present in a second sequence at a position corresponding to a specified amino acid residue in a first sequence which is the same position as the position in the first sequence when the two sequences are aligned to allow for maximum sequence identity between the two sequences.

As used herein, “consists essentially of” or “consisting essentially of” means that a polypeptide may have additional features or elements beyond those described, provided that such additional features or elements do not materially affect the ability of the antibody or antibody fragment to have the recited binding specificity. The antibody or antibody fragments comprising the polypeptides may have additional features or elements that do not interfere with the ability of the antibody or antibody fragments to bind to its target and exhibit its functional activity, e.g., disrupting or preventing bacterial adhesion to a mannose-coated surface. Such modifications may be introduced into the amino acid sequence in order to reduce the immunogenicity of the antibody. For example, a polypeptide consisting essentially of a specified sequence may contain one, two, three, four, five or more additional, deleted or substituted amino acids, at either end or at both ends of the sequence provided that these amino acids do not interfere with, inhibit, block or interrupt the role of the antibody or fragment in binding to its target and exhibiting its biological activity.

As used herein, a “heterologous” sequence or a “heterologous” molecule refers to a moiety not naturally occurring in conjunction with a recited sequence or molecule. Representative examples of the heterologous molecule include, but are not limited to, a polypeptide, antibody, epitope, polynucleotide, small molecule or drug. Such heterologous moieties can be useful for improving solubility, delivery, immunogenicity, efficacy, detection, or identification of the recited sequence or molecule. In some embodiments, the heterologous sequence is inert or an unrelated sequence.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0,9%) saline.

Compositions comprising such carriers are formulated by well-known conventional methods (see, for example, Remington's Pharmaceutical Sciences, 18th edition, (Remington and Gennaro 1990)).

As used herein, to “prevent” or “treat” a condition means to decrease or inhibit symptoms indicative of the condition or to delay the onset or reduce the severity of the condition.

As used herein, “adjuvant” includes those adjuvants commonly used in the art to facilitate an immune response. In some embodiments, such as with the use of a polynucleotide vaccine, an adjuvant such as a helper peptide or cytokine can be provided via a polynucleotide encoding the adjuvant.

As used herein, “a” or “an” means at least one, unless clearly indicated otherwise.

As used herein, the terms “comprise” or “include”, or variations such as “comprises” or “comprising”, “includes” or “including” mean the inclusion of a recited item or group of items, but not the exclusion of any other item or group of items.

### Constructs and Compositions of the Invention

The invention provides, in one embodiment, a polynucleotide construct encoding a chimeric cellular receptor. In one embodiment, the construct comprises nucleic acid sequences encoding, in operable linkage, a myristoylation sequence (Myr), a dimerizer domain, and an intracellular portion of the TLR4 receptor. In some embodiments, the construct further comprises a sequence that enables expression of a separate reporter molecule controlled by the same promoter. Examples of such a sequence include, but are not limited to, a ribosome skipping sequence and a cleavage sequence. One example of a reporter molecule is green fluorescent protein (GFP). In one embodiment, the construct is free of sequences encoding an extracellular portion of the TLR4 receptor. In preferred embodiments, the construct is sufficiently free of sequences encoding an extracellular portion of the TLR4 receptor that no extracellular ligands will bind. In one embodiment, the dimerizer domain is phenylalanine 36 to valine point mutation (F36V) derived from a mutated version of the endogenous FKBP12 protein.

In one embodiment, the nucleic acid sequences encoding the myristoylation sequence (Myr), the dimerizer domain, and the intracellular portion of the TLR4 receptor comprises a nucleotide sequence encoding an amino acid sequence having at least 90% sequence identity to the amino acid sequence of:

(SEQ ID NO: 1)
MGSSKSKPKDPSQRLEGVQVETISPGDGRTFPKRGQTCVVHYTGMLEDGK
KVDSSRDRNKPFKFMLGKQEVIRGWEEGVAQMSVGQRAKLTISPDYAYGA
TGHPGIIPPHATLVFDVELLKLEGSIAGCKKYSRGESIYDAFVIYSSQNE
DWVRNELVKNLEEGVPRFHLCLHYRDFIPGVAIAANIIQEGFHKSRKVIV
VVSRHFIQSRWCIFEYEIAQTWQFLSSRSGIIFIVLEKVEKSLLRQQVEL
YRLLSRNTYLEWEDNPLGRHIFWRRLKKALLDGKASNPEQTAEEEQETAT
WT.

In one embodiment, the nucleic acid sequences encoding the myristoylation sequence (Myr), the dimerizer domain, the intracellular portion of the TLR4 receptor, the ribosome skipping sequence, and the green fluorescent protein comprises a nucleotide sequence encoding an amino acid sequence having at least 90% sequence identity to the amino acid sequence of:

(SEQ ID NO: 2)
M G S S K S K P K D P S Q R L E G V Q V E T I S P
G D G R T F P K R G Q T C V V H Y T G M L E D G K
K V D S S R D R N K P F K F M L G K Q E V I R G W
E E G V A Q M S V G Q R A K L T I S P D Y A Y G A
T G H P G I I P P H A T L V F D V E L L K L E G S
I A G C K K Y S R G E S I Y D A F V I Y S S Q N E
D W V R N E L V K N L E E G V P R F H L C L H Y R
D F I P G V A I A A N I I Q E G F H K S R K V I V
V V S R H F I Q S R W C I F E Y E I A Q T W Q F L
S S R S G I I F I V L E K V E K S L L R Q Q V E L
Y R L L S R N T Y L E W E D N P L G R H I F W R R
L K K A L L D G K A S N P E Q T A E E E Q E T A T
W T E F E G S A A A E G R G S L L T C G D V E E N
P G P S G M E S D E S G L P A M E I E C R I T G T
L N G V E F E L V G G G E G T P K Q G R M T N K M
K S T K G A L T F S P Y L L S H V M G Y G F Y H F
G T Y P S G Y E N P F L H A I N N G G Y T N T R I
E K Y E D G G V L H V S F S Y R Y E A G R V I G D
F K V V G T G F P E D S V I F T D K I I R S N A T
V E H L H P M G D N V L V G S F A R T F S L R D G
G Y Y S F V V D S H M H F K S A I H P S I L Q N G
G P M F A F R R V E E L H S N T E L G I V E Y Q H
A F K T P I A F.

In one embodiment, the intracellular portion of the TLR4 receptor comprises an amino acid sequence having at least 90% sequence identity to the amino acid sequence of LAGCIKYGRGENIYDAFVIYSSQDEDWVRNELVKNLEEGVPPFQLCLHYRDFIPGVAIAA NIIHEGFHKSRKVIVVVSQHFIQSRWCIFEYEIAQTWQFLSSRAGIIFIVLQKVEKTLLRQQ VELYRLLSRNTYLEWEDSVLGRHIFWRRLRKALLDGKSWNPEGTVGTGCNWQEATSI (SEQ ID NO: 6). In some embodiments, the intracellular portion of the TLR4 receptor includes one or more of the following mutations: P714A, S744A, R745A, C747S, Y751A, E752A, E775A, K776S, Q792A, N792 A, Y794A, E796A, and E798A. Representative examples of the intracellular portion of the TLR4 receptor include, but are not limited to, polypeptides comprising an amino acid sequence selected from:

(SEQ ID NO: 7)
LAGCIKYGRGENIYDAFVIYSSQDEDWVRNELVKNLEEGVPPFQLCLHYR
DFIAGVAIAANIIHEGFHKSRKVIVVVSQHFIQSRWCIFEYEIAQTWQFL
SSRAGIIFIVLQKVEKTLLRQQVELYRLLSRNTYLEWEDSVLGRHIFWRR
LRKALLDGKSWNPEGTVGTGCNWQEATSI;
(SEQ ID NO: 8)
LAGCIKYGRGENIYDAFVIYSSQDEDWVRNELVKNLEEGVPPFQLCLHYR
DFIPGVAIAA NIIHEGFHKSRKVIVVVSQHFIQARWCIFEYEIAQTWQF
LSSRAGIIFIVLQKVEKTLLRQQVELYRLLSRNTYLEWEDSVLGRHIFWR
RLRKALLDGKSWNPEGTVGTGCNWQEATSI;
(SEQ ID NO: 9)
LAGCIKYGRGENIYDAFVIYSSQDEDWVRNELVKNLEEGVPPFQLCLHYR
DFIPGVAIAA NIIHEGFHKSRKVIVVVSQHFIQSAWCIFEYEIAQTWQF
LSSRAGIIFIVLQKVEKTLLRQQVELYRLLSRNTYLEWEDSVLGRHIFWR
RLRKALLDGKSWNPEGTVGTGCNWQEATSI;
(SEQ ID NO: 10)
LAGCIKYGRGENIYDAFVIYSSQDEDWVRNELVKNLEEGVPPFQLCLHYR
DFIPGVAIAA NIIHEGFHKSRKVIVVVSQHFIQSRWSIFEYEIAQTWQF
LSSRAGIIFIVLQKVEKTLLRQQVELYRLLSRNTYLEWEDSVLGRHIFWR
RLRKALLDGKSWNPEGTVGTGCNWQEATSI;
(SEQ ID NO: 11)
LAGCIKYGRGENIYDAFVIYSSQDEDWVRNELVKNLEEGVPPFQLCLHYR
DFIPGVAIAA NIIHEGFHKSRKVIVVVSQHFIQSRWCIFEAEIAQTWQF
LSSRAGIIFIVLQKVEKTLLRQQVELYRLLSRNTYLEWEDSVLGRHIFWR
RLRKALLDGKSWNPEGTVGTGCNWQEATSI;
(SEQ ID NO: 12)
LAGCIKYGRGENIYDAFVIYSSQDEDWVRNELVKNLEEGVPPFQLCLHYR
DFIPGVAIAA NIIHEGFHKSRKVIVVVSQHFIQSRWCIFEYAIAQTWQF
LSSRAGIIFIVLQKVEKTLLRQQVELYRLLSRNTYLEWEDSVLGRHIFWR
RLRKALLDGKSWNPEGTVGTGCNWQEATSI;
(SEQ ID NO: 13)
LAGCIKYGRGENIYDAFVIYSSQDEDWVRNELVKNLEEGVPPFQLCLHYR
DFIPGVAIAA NIIHEGFHKSRKVIVVVSQHFIQSRWCIFEYEIAQTWQF
LSSRAGIIFIVLQKVAKILLRQQVELYRLLSRNTYLEWEDSVLGRHIFWR
RLRKALLDGKSWNPEGTVGTGCNWQEATSI;
(SEQ ID NO: 14)
LAGCIKYGRGENIYDAFVIYSSQDEDWVRNELVKNLEEGVPPFQLCLHYR
DFIPGVAIAA NIIHEGFHKSRKVIVVVSQHFIQSRWCIFEYEIAQTWQF
LSSRAGIIFIVLQKVESTLLRQQVELYRLLSRNTYLEWEDSVLGRHIFWR
RLRKALLDGKSWNPEGTVGTGCNWQEATSI;
(SEQ ID NO: 15)
LAGCIKYGRGENIYDAFVIYSSQDEDWVRNELVKNLEEGVPPFQLCLHYR
DFIPGVAIAA NIIHEGFHKSRKVIVVVSQHFIQSRWCIFEYEIAQTWQF
LSSRAGIIFIVLQKVEKTLLRAQVELYRLLSRNTYLEWEDSVLGRHIFWR
RLRKALLDGKSWNPEGTVGTGCNWQEATSI;
(SEQ ID NO: 16)
LAGCIKYGRGENIYDAFVIYSSQDEDWVRNELVKNLEEGVPPFQLCLHYR
DFIPGVAIAA NIIHEGFHKSRKVIVVVSQHFIQSRWCIFEYEIAQTWQF
LSSRAGIIFIVLQKVEKTLLRQQVELYRLLSRATYLEWEDSVLGRHIFWR
RLRKALLDGKSWNPEGTVGTGCNWQEATSI;
(SEQ ID NO: 17)
LAGCIKYGRGENIYDAFVIYSSQDEDWVRNELVKNLEEGVPPFQLCLHYR
DFIPGVAIAA NIIHEGFHKSRKVIVVVSQHFIQSRWCIFEYEIAQTWQF
LSSRAGIIFIVLQKVEKTLLRQQVELYRLLSRNTALEWEDSVLGRHIFWR
RLRKALLDGKSWNPEGTVGTGCNWQEATSI;
(SEQ ID NO: 18)
LAGCIKYGRGENIYDAFVIYSSQDEDWVRNELVKNLEEGVPPFQLCLHYR
DFIPGVAIAA NIIHEGFHKSRKVIVVVSQHFIQSRWCIFEYEIAQTWQF
LSSRAGIIFIVLQKVEKTLLRQQVELYRLLSRNTYLAWEDSVLGRHIFWR
RLRKALLDGKSWNPEGTVGTGCNWQEATSI;
and
(SEQ ID NO: 19)
LAGCIKYGRGENIYDAFVIYSSQDEDWVRNELVKNLEEGVPPFQLCLHYR
DFIPGVAIAA NIIHEGFHKSRKVIVVVSQHFIQSRWCIFEYEIAQTWQF
LSSRAGIIFIVLQKVEKTLLRQQVELYRLLSRNTYLEWADSVLGRHIFWR
RLRKALLDGKSWNPEGTVGTGCNWQEATSI.

The construct described herein was made using a TLR4-Sport6 Vector (Open Biosystems):

The construct begins with TLR4 (particularly amino acids 675-835; transmembrane helices underlined; TIR cytoplasmic domain in bold and highlighted; a linker sequence is added between them; the proline that is both bold and underlined is needed for signaling):

(SEQ ID NO: 21)
MNPPWLLARTLIMALFFSCLTPGSLNPCIEVVPNITYQCMDQKL
SKVPDDIPSSTKNIDLSFNPLKILKSYSFSNFSELQWLDLSRCEIETIEDKAWHGLHH
LSNLILTGNPIQSFSPGSFSGLTSLENLVAVETKLASLESFPIGQLITLKKLNVAHNF
IHSCKLPAYFSNLTNLVHVDLSYNYIQTITVNDLQFLRENPQVNLSLDISLNPIDFIQ
DQAFQGIKLHELTLRGNFNSSNIMKTCLQNLAGLHVHRLILGEFKDERNLEIFEPSIM
EGLCDVTIDEFRLTHTNDFSDDIVKFHCLANVSAMSLAGVSIKYLEDVPKHFKWQSLS
IIRCQLKQFPTLDLPFLKSLTLTMNKGSISFKKVALPSLSYLDLSRNALSFSGCCSYS
DLGTNSLRHLDLSFNGAIIMSANFMGLEELQHLDFQHSTLKRVTEFSAFLSLEKLLYL
DISYTNTKIDFDGIFLGLTSLNTLKMAGNSFKDNTLSNVFANTTNLTFLDLSKCQLEQ
ISWGVFDTLHRLQLLNMSHNNLLFLDSSHYNQLYSLSTLDCSFNRIETSKGILQHFPK
SLAFFNLTNNSVACICEHQKFLQWVKDQKQFLVNVEQMTCATPVEMNTSLVLDFNNST

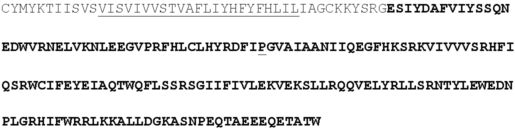

Nucleotide sequence for cTLR4 (by uniprot.org; 2207-2738; Xho 1 is underlined):

(SEQ ID NO: 22)
5′-ATTGCTGGCTGTAAAAAGTACAGCAGAGGAGAAAGCATCTATGATGC
ATTTGTGATCTACTCGAGTCAGAATGAGGACTGGGTGAGAAATGAGCTGG
TAAAGAATTTAGAAGAAGGAGTGCCCCGCTTTCACCTCTGCCTTCACTAC
AGAGACTTTATTCCTGGTGTAGCCATTGCTGCCAACATCATCCAGGAAGG
CTTCCACAAGAGCCGGAAGGTTATTGTGGTAGTGTCTAGACACTTTATTC
AGAGCCGTTGGTGTATCTTTGAATATGAGATTGCTCAAACATGGCAGTTT
CTGAGCAGCCGCTCTGGCATCATCTTCATTGTCCTTGAGAAGGTTGAGAA
GTCCCTGCTGAGGCAGCAGGTGGAATTGTATCGCCTTCTTAGCAGAAACA
CCTACCTGGAATGGGAGGACAATCCTCTGGGGAGGCACATCTTCTGGAGA
AGACTTAAAAATGCCCTATTGGATGGAAAAGCCTCGAATCCTGAGCAAAC
AGCAGAGGAAGAACAAGAAACGGCAACTTGGACC-3′.

Final engineered construct DNA sequence (initial lowercase sequence is EF1 promoter; followed by, in uppercase, Kozak sequence, myristoylation domain (underlined), F36V domain (underlined), cTLR4 sequence (underlined), and in lowercase and underlined, T2A sequence and copGFP; SEQ ID NO: 3):

aaggatctgcgatcgctccggtgcccgtcagtgggcagagcgcacatcgc
ccacagtccccgagaagttggggggaggggtcggcaattgaacgggtgcc
tagagaaggtggcgcggggtaaactgggaaagtgatgtcgtgtactggct
ccgcctttttcccgagggtgggggagaaccgtatataagtgcagtagtcg
ccgtgaacgttctttttcgcaacgggtttgccgccagaacacagctgaag
cttcgaggggctcgcatctctccttcacgcgcccgccgccctacctgagg
ccgccatccacgccggttgagtcgcgttctgccgcctcccgcctgtggtg
cctcctgaactgcgtccgccgtctaggtaagtttaaagctcaggtcgaga
ccgggcctttgtccggcgctcccttggagcctacctagactcagccggct
ctccacgctttgcctgaccctgcttgctcaactctacgtctttgtttcgt
tttctgttctgcgccgttacagatccaagctgtgaccggcgcctactcta
gagctagcGCCACCATGGGGAGTAGCAAGAGCAAGCCTAAGGACCCCAGC
CAGCGCCTCGAGGGCGTGCAGGTGGAGACTATCTCCCCAGGAGACGGGCG
CACCTTCCCCAAGCGCGGCCAGACCTGCGTGGTGCACTACACCGGGATGC
TTGAAGATGGAAAGAAAGTTGATTCCTCCCGGGACAGAAACAAGCCCTTT
AAGTTTATGCTAGGCAAGCAGGAGGTGATCCGAGGCTGGGAAGAAGGGGT
TGCCCAGATGAGTGTGGGTCAGAGAGCCAAACTGACTATATCTCCAGATT
ATGCCTATGGTGCCACTGGGCACCCAGGCATCATCCCACCACATGCCACT
CTCGTCTTCGATGTGGAGCTTCTAAAACTGGAAGGATCCATTGCTGGCTG
TAAAAAGTACAGCAGAGGAGAAAGCATCTATGATGCATTTGTGATCTACT
CGAGTCAGAATGAGGACTGGGTGAGAAATGAGCTAGTAAAGAATTTAGAA
GAAGGAGTGCCCCGCTTTCACCTCTGCCTTCACTACAGAGACTTTATTCC
TGGTGTAGCCATTGCTGCCAACATCATCCAGGAAGGCTTCCACAAGAGCC
GGAAGGTTATTGTGGTAGTGTCTAGACACTTTATTCAGAGCCGTTGGTGT
ATCTTTGAATATGAGATTGCTCAAACATGGCAGTTTCTGAGCAGCCGCTC
TGGCATCATCTTCATTGTCCTTGAGAAGGTTGAGAAGTCCCTGCTGAGGC
AGCAGGTGGAATTGTATCGCCTTCTTAGCAGAAACACCTACCTGGAATGG
GAGGACAATCCTCTGGGGAGGCACATCTTCTGGAGAAGACTTAAAAAGGC
CCTATTGGATGGAAAAGCCTCGAATCCTGAGCAAACAGCAGAGGAAGAAC
AAGAAACGGCAACTTGGACCgaattcgaaggatccgcggccgctgagggc
agaggaagtcttctaacatgcggtgacgtggaggagaatcccggcccttc
cggaatggagagcgacgagagcggcctgcccgccatggagatcgagtgcc
gcatcaccggcaccctgaacggcgtggagttcgagctggtgggcggcgga
gagggcacccccaagcagggccgcatgaccaacaagatgaagagcaccaa
aggcgccctgaccttcagcccctacctgctgagccacgtgatgggctacg
gcttctaccacttcggcacctaccccagcggctacgagaaccccttcctg
cacgccatcaacaacggcggctacaccaacacccgcatcgagaagtacga
ggacggcggcgtgctgcacgtgagcttcagctaccgctacgaggccggcc
gcgtgatcggcgacttcaaggtggtgggcaccggcttccccgaggacagc
gtgatcttcaccgacaagatcatccgcagcaacgccaccgtggagcacct
gcaccccatgggcgataacgtgctggtgggcagcttcgcccgcaccttca
gcctgcgcgacggcggctactacagcttcgtggtggacagccacatgcac
ttcaagagcgccatccaccccagcatcctgcagaacgggggccccatgtt
cgccttccgccgcgtggaggagctgcacagcaacaccgagctgggcatcg
tggagtaccagcacgccttcaagacccccatcgccttcgcc.

The protein sequence translated and labeled (SEQ ID NO: 4):

MGSSKSKPKDPSQRL (myristoylation domain)
EGVQVETISPGDGRTFPKRGQTCVVHYTGMLEDGKKVDSSRDRNKPFKFM
LGKQEVIRGWEEGVAQMSVGQRAKLTISPDYAYGATGHPGIIPPHATLVF
DVELLKLE (F36V domain)
GSIAGCKKYSRGESIYDAFVIYSSQNEDWVRNELVKNLEEGVPRFHLCLH
YRDFIPGVAIAANIIQEGFHKSRKVIVVVSRHFIQSRWCIFEYEIAQTWQ
FLSSRSGIIFIVLEKVEKSLLRQQVELYRLLSRNTYLEWEDNPLGRHIFW
RRLKKALLDGKASNPEQTAEEEQETATWT (cTLR4 domain)
EFEGSAAA (spacer) EGRGSLLTCGDVEENPGP (T2A se-
quence) SGMESDESG (spacer) LPAMEIECRITGTLNGVEFELVG
GGEGTPKQGRMTNKMKSTKGALTFSPYLLSHVMGYGFYHFGTYPSGYENP
FLHAINNGGYTNTRIEKYEDGGVLHVSFSYRYEAGRVIGDFKVVGTGFPE
DSVIFTDKIIRSNATVEHLHPMGDNVLVGSFARTFSLRDGGYYSFVVDSH
MHFKSAIHPSILQNGGPMFAFRRVEELHSNTELGIVEYQHAFKTPIAF
(copGFP) A.

In one embodiment, the invention provides a composition comprising a construct of the invention. In one embodiment, the construct encodes the myristoylation domain, the F36V domain, and the cTLR4 domain. Optionally, the construct further comprises the T2A sequence and copGFP, or reporter sequence. In another embodiment, the invention provides a composition comprising a cell transfected with a construct of the invention. Typically, the cell is a monocyte.

In one embodiment, the composition is a pharmaceutical composition. The composition can comprise a therapeutically or prophylactically effective amount of construct, or engineered monocyte transfected with same, of the invention. An effective amount is an amount sufficient to achieve pro-inflammatory macrophage activation, or to alleviate symptoms of a condition, disease, or infection. In some embodiments, the composition of the invention further comprises a carrier. The carrier can be a pharmaceutically acceptable carrier, or other carrier that facilitates use of the composition.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes via known methods.

### Administration of the Compositions

Treatment includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Typical patients or subjects are human.

Compositions are typically administered in vivo via parenteral (e.g, intravenous, subcutaneous, and intramuscular) or other traditional direct routes, such as buccal/sublingual, rectal, oral, nasal, topical, (such as transdermal and ophthalmic), vaginal, pulmonary, intraarterial, intraperitoneal, intraocular, or intranasal routes or directly into a specific tissue, such as by implantation into a target organ, injection into a target tissue, or introduction of a scaffold to a target site.

The compositions are administered in any suitable manner, often with pharmaceutically acceptable carriers. Suitable methods of administering cells in the context of the present invention to a patient are available, and, although more than one route can be used to administer a particular composition, a particular route can often provide a more immediate and more effective reaction than another route.

The dose administered to a patient, in the context of the present invention should be sufficient to effect a beneficial therapeutic response in the patient over time, or to inhibit infection or disease due to infection. Thus, the composition is administered to a patient in an amount sufficient to alleviate, reduce, cure or at least partially arrest symptoms and/or complications from the disease or infection, An amount adequate to accomplish this is defined as a “therapeutically effective dose.”

The dose will be determined by the activity of the composition produced and the condition of the patient, as well as the body weight or surface areas of the patient to be treated. The size of the dose also will be determined by the existence, nature, and extent of any adverse side effects that accompany the administration of a particular composition in a particular patient. In determining the effective amount of the composition to be administered in the treatment or prophylaxis of disease, the physician needs to evaluate the progression of the disease, and any treatment-related toxicity.

### Methods and Uses of the Invention

The invention further provides a method of producing a genetically engineered monocyte. In one embodiment, the method comprises the steps of: (a) contacting a monocyte with a construct as described herein under conditions sufficient to transfect the construct into the monocyte; and (b) culturing the monocyte transfected in step (a). The genetically engineered monocytes express the chimeric cellular receptor able to dimerize upon addition of a Chemical Inducer of Dimerization (CID) synthetic ligand, which dimerization activates signaling pathways independently of endogenous physiological ligands, thereby differentiating the transfected monocyte into a macrophage. In one embodiment, the CID synthetic ligand is a recombinant FK506 molecule. In one embodiment, the recombinant FK506 molecule is AP20187.

Also provided is a genetically engineered monocyte produced in accordance with the methods described herein, as well as a pharmaceutical composition comprising a genetically engineered monocyte of the invention. In some embodiments, the composition further comprises a CID synthetic ligand. In one embodiment, the CID synthetic ligand is a recombinant FK506 molecule, such as, for example, AP20187.

The invention additionally provides a method of reversibly inducing pro-inflammatory macrophages in a subject. In one embodiment, the method comprises: (a) administering the composition of claim 11 to a subject; and (b) administering a CID synthetic ligand to the subject. The CID synthetic ligand activates the genetically engineered monocytes. In one embodiment, the method further comprises: (c) withdrawing administration of the CID synthetic ligand and/or administering a washout ligand, thereby reversing the activation of the genetically engineered monocytes. One example of a washout ligand for use with AP20187 is B/B Washout Ligand (Clontech). The administering of step (a) can be via means suitable to the particular patient and treatment objective, such as by implantation into a target organ, injection into a target tissue, introduction of a scaffold to a target site, or intravenous administration, In some embodiments, the genetically engineered monocyte is pre-treated with a CID synthetic ligand prior to the administering of step (a). In such embodiments, pre-polarized cells are administered to the subject, thereby jump-starting the process. The CID synthetic ligand is administered subsequently to maintain the cells in a polarized state in vivo

The invention provides methods of treating conditions that would benefit from selective inducement of pro-inflammatory macrophages. In one embodiment, the invention provides a method of treating a fibrotic disease comprising administering to a subject in need thereof the pharmaceutical composition of the invention and a CID synthetic ligand. Representative examples of fibrotic disease include, but are not limited to, those selected from the group consisting of pulmonary fibrosis, cardiac fibrosis, and foreign body reaction. In another embodiment, the invention provides a method of treating an inflammatory disease comprising administering to a subject in need thereof the pharmaceutical composition of the invention and a CID synthetic ligand. In one embodiment, the inflammatory disease is a chronic inflammatory disease. Representative examples of chronic inflammatory disease include, but are not limited to, those selected from the group consisting of atherosclerosis and rheumatoid arthritis. In yet another embodiment, the invention provides a method of treating cancer inflammation comprising administering to a subject in need thereof the pharmaceutical composition of the invention and a CID synthetic ligand.

### Kits

For use in the methods described herein, kits are also within the scope of the invention. Such kits can comprise a package or container that is compartmentalized to receive one or more containers such as vials, tubes, and the like, each of the container(s) comprising one of the separate elements (e.g., constructs, cells, dimerizing agent, matrigel, scaffold) to be used in the method. Typically, the kit comprises one or more constructs of the invention. The kit further comprises one or more containers, with one or more constructs stored in the containers. The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific therapeutic or non-therapeutic application, and can also indicate directions for use. Directions and or other information can also be included on an insert which is included with the kit.

### EXAMPLES

The following examples are presented to illustrate the present invention and to assist one of ordinary skill in making and using the same. The examples are not intended in any way to otherwise limit the scope of the invention.

### Example 1

### Engineering Macrophages to Control the Inflammatory Response and Angiogenesis

The physiological innate inflammatory response requires a highly orchestrated series of events characterized by four basic phases: reaction, regrowth, remodeling, and resolution.[1] During the course of this healing process, MΦ play an active role in secreting chemokines and cytokines that direct the recruitment and egress of various immune cell types at the injured site. The functional MΦ phenotype depends on the microenvironment of the injured site and alters accordingly during the normal process of healing.[2] However, dysregulation of the MΦ phenotype can lead to a non-ideal healing outcome.

Monocytes are the precursor cells to MΦ, which are a main inflammatory cell type and are known to be key players in the inflammatory response. When activated, MΦ are classified into two major phenotypes that can be broadly defined as: pro-inflammatory MΦ and pro-healing MΦ. In literature, pro-inflammatory MΦ are often denoted as “classically activated” or “M1” and pro-healing MΦ are denoted as “alternatively activated” or “M2.” However, these two major MΦ phenotypes are the two extremes on the phenotype scale, as intermediate macrophage types also exist.[3] During the inflammatory reaction, M1 MΦ are the first to arrive at the inflammation site and this MΦ population subsequently shifts to a less inflammatory pro-healing M2 MΦ population during the repair phase. Pro-inflammatory MΦ release inflammatory cytokines, such as TNFα and IL-6 as well as produce reactive oxygen species (ROS).[4, 5] In comparison, the pro-healing MΦ phenotype has been shown to produce cytokines, such as IL-10 and TGFβ1, which are markers that can decrease the pro-inflammatory response and promote healing and fibrosis.[3]

Angiogenesis, or the formation of new blood vessels, is a critical step in the wound healing process. In the course of the inflammation response, pro-inflammatory MΦ secrete TNFα and IFN-γ, which regulate expression of adhesion molecules on EC. These cytokines promote leukocyte adhesion to EC and extravasation into tissues by increasing expression of both cell surface and soluble forms of vascular cell adhesion protein 1 (VCAM-1) and intercellular adhesion molecule 1 (ICAM-1).[6-9] These two adhesion molecules belong to the immunoglobulin superfamily group and have been implicated, along with integrins, in pro-angiogenic processes.[10, 11]

This study utilizes engineered pro-inflammatory M1-like cells to investigate EC activation. The engineered cells were designed by using the chemical inducer of dimerization (CID) technology to induce activation of the TLR4 receptor independent of the lipopolysaccharides (LPS) exogenous ligand, which is a well-established inducer of the M1 MΦ phenotype.[12] The CID technology has been used in several other groups to control a variety of cell signaling pathways.[13-17] For this system to function, the intracellular domain of a desired receptor is fused to a F36V protein, which is a mutated version of the FKBP12 protein. This F36V version has a binding site for a cell permeable CID drug. When CID drug is present, two F36V proteins will dimerize, bringing the desired intracellular domains in close enough proximity to activate receptor-specific pathways. These cells can be activated by the exposure to CID drug and be deactivated by the withdrawal of CID drug. Currently in literature there have been no cellular engineering approaches to control or modulate MΦ polarization to determine ideal healing conditions. Having the ability to control MΦ polarization during and after an inflammatory response could potentially allow for the manipulation of the host response and the optimal healing of multiple inflammatory conditions.

### Methods and Materials:

### Reagents and Antibodies

The monoclonal anti-human/mouse/rat FKBP12 antibody was purchased from Thermo Scientific. The following antibodies were purchased from Cell Signaling: p44/42 MAPK, Phospho-p44/42 MAPK, IRF3 and Phospho-IRF3. The anti-iNOS/NOS type II antibody was purchased from BD Biosciences. The anti-mouse CD106 (VCAM-1) PE, the anti-mouse CD54 (ICAM-1) PE, the rat IgG2b isotype, and the anti-mouse TNFα antibodies were purchased from eBioscience. The HRP-conjugated goat-anti-rabbit antibody was obtained from Jackson ImmunoResearch Laboratories, Inc. and the HRP-conjugated goat-anti-mouse antibody was obtained from Life Technologies. LPS and recombinant mouse TNFα was purchased from Sigma and recombinant mouse IL-4 was purchased from eBioscience. AP20187 (CID drug) was purchased from Clontech. Lipofectamine 2000 was purchased from Invitrogen. The Dual-Luciferase® reporter assay system was obtained from Promega Corporation.

Plasmid Construction of cTLR4

The mouse Sport6-TLR4 vector was purchased from Open Biosystems. The cytoplasmic portion of TLR4 (cTLR4) was amplified (mRNA base pairs 2207-2748) and inserted into a pBluescript II KS+ vector with an existing myristolation domain and engineered F36V domain (pBluescript-Myr-F36V) (55) following BamHl and EcoRV restriction enzyme (RE) cuts. PCR products were gel purified using a QIAEX II gel extraction kit (Qiagen) before ligations were performed. This resulted in a pBluescript-Myr-F36V-cTLR4 construct. The pCDH-EF1α-MCS-T2A-copGFP lentiviral cDNA and expression vector was purchased from System Biosciences. This vector was cut in the MCS with both NheI and EcoRI, and a PCR amplified portion of the Myr-F36V-cTLR4 sequence was ligated into this site within the pCDH-EF1α-MCS-T2A-copGFP vector (7.26 kb). This resulted in the final cTLR4 lentiviral plasmid: pCDH-EF1α-Myr-F36V-cTLR4-T2A-copGFP (8.18 kb).

Cell Transduction of cTLR4 Lentiviral Constructs

We utilized a 3rd generation lentiviral vector, pCDH (System Biosciences), carrying the cTLR4 gene under the control of the EF-1α promoter. For stable lentiviral transductions, 5×106 HEK293T packaging cells were seeded in 10-cm cell culture dishes that were previously coated with 50 ug/mL poly-D-lysine hydrobromide (Sigma). Culture medium was changed just prior to transduction. In total, 12 μg plasmid DNA was used for each 10-cm dish (2.8 μg transfer vector (cTLR4), 0.9 μg pSL3 (vesicular stomatitis virus G envelope), 5.4 μg pSL4 (HIV-1 gag/pol packing genes), and 2.8 μg pSL5 (rev gene required for HIV-1 envelope protein expression). DNA and Lipofectamine 2000™ (Life Technologies) were diluted in Opti-MEM® medium (Gibco) separately. After a 5 minute incubation, DNA and lipofectamine were combined and incubated for 20 minutes at room temperature. The complexes were then added, drop-wise, to cell dishes with 8 mL growth media and medium was replaced after 14-16 hours. Virus supernatant was collected following an additional 48 hours by filtering through a 0.45 μm filter. Filtered virus supernatant was then added either directly or in concentrated form to previously plated RAW264.7 cells (5×105 cells per well) in 6-well plates. Cells were then sorted for GFP expression to acquire >90% transduction efficiency.

### Cell Culture

RAW264.7 and bEnd.3 cells were obtained from ATCC. RAW264.7 and bEnd.3 cells were cultured in DMEM medium from Invitrogen containing 10% (v/v) heat-inactivated FBS and 100 U/ml pen/strep (Invitrogen) and incubated at 37° C. with 5% CO2.

### Western Blotting

Protein from RAW264.7, MΦ-T2A (vector control cells), and MΦ-cTLR4 cell monolayers were extracted by lysis in Laemmli buffer containing 1× Halt Protease Inhibitor cocktail (Thermo Scientific). Following lysis, samples were boiled and protein concentration was determined by performing a BCA assay from Thermo Scientific. Samples (10-30 μg of lysates) were run on 4-20% Mini-PROTEAN® TGX precast polyacrylamide gels (Bio-Rad). Protein from gels were transferred onto PVDF membranes and probed with the appropriate primary antibody overnight. Membranes were washed between each antibody incubation and subsequently probed with the appropriate HRP-conjugated secondary antibody (Life Technologies). The Clarity Western ECL Substrate (Bio-Rad) was used to detect bands.

### Cytokine Profile

We tested IL-6 and TNFα concentrations in supernatants of transduced RAW264.7 cells in vitro. Briefly, MΦ-cTLR4 cells (1×106) were plated in each well of a 6-well plate and treated with vehicle (100% EtOH), LPS (100 ng/mL), CID drug (50 nM), or left untreated in DMEM without serum. Supernatants were collected and tested using the mouse IL-6 ELISA Ready-SET-Go! and the mouse TNFα ELISA Ready-SET-Go! Kits (eBioscience) according to the manufacturer's instructions. Plates were read at 450 nM with a 570 nM wavelength subtraction, normalized to standard solutions, and concentrations (pg/mL) were calculated.

### Luciferase Assay

Two hundred thousand cells/well were seeded in 24-well plates. The following day each well was transfected with a total of 0.8 μg plasmid DNA, which consisted of a 20:1 ratio of pBllX-LUC (NFκB reporter construct):pRL (Renilla luciferase construct). The promoterless pGL4.10 vector was also transfected in a 20:1 ratio of pGL4.10:pRL, as a control. Transfections of each well were performed with 2 μL Lipofectamine 2000 (Invitrogen). The following morning transfection reagents were replaced with fresh serum-free medium and treated with either vehicle (100% EtOH) or CID drug (50 nM) for 4 hours. Cell lysate was harvested and luciferase activity was measured using a Dual-Luciferase® reporter assay kit (Promega) according to manufacturer's instructions. All groups were normalized to Renilla luciferase.

### Endothelial Cell Activation

MΦ-cTRL4 conditioned media, following a 6 hour treatment in 6-well plates (1×106 cells/well), was transferred to plated bEnd.3 cells in a 12-well plate (0.2×106 cells/well). Before media transfer, TNFα neutralizing and IgG isotype antibody (1 μg/mL) were incubated in media for 15 minutes. Media was then added to bEnd.3 cells for 12 hours. Following incubation, bEnd.3 cells were trypsinized and stained for ICAM-1 and VCAM-1. Cell cytometry was performed on a FACSCanto II Cell Analyzer (BD Biosciences) equipped with 488 nm and 647 nm lasers. Typically, 10,000 cells were analyzed per sample. Experiments were repeated at least three times. Non-specific staining was evaluated using a monoclonal antibody for IgG2b and IgG2a (eBioscience).

### Statistical Analysis

Results are expressed as mean±SE unless otherwise specified. Significance between groups was determined by ANOVA and p-values less than 0.05 were considered significant.

### Results

Engineering M101 -cTLR4 Pro-Inflarnrnatory Macrophages

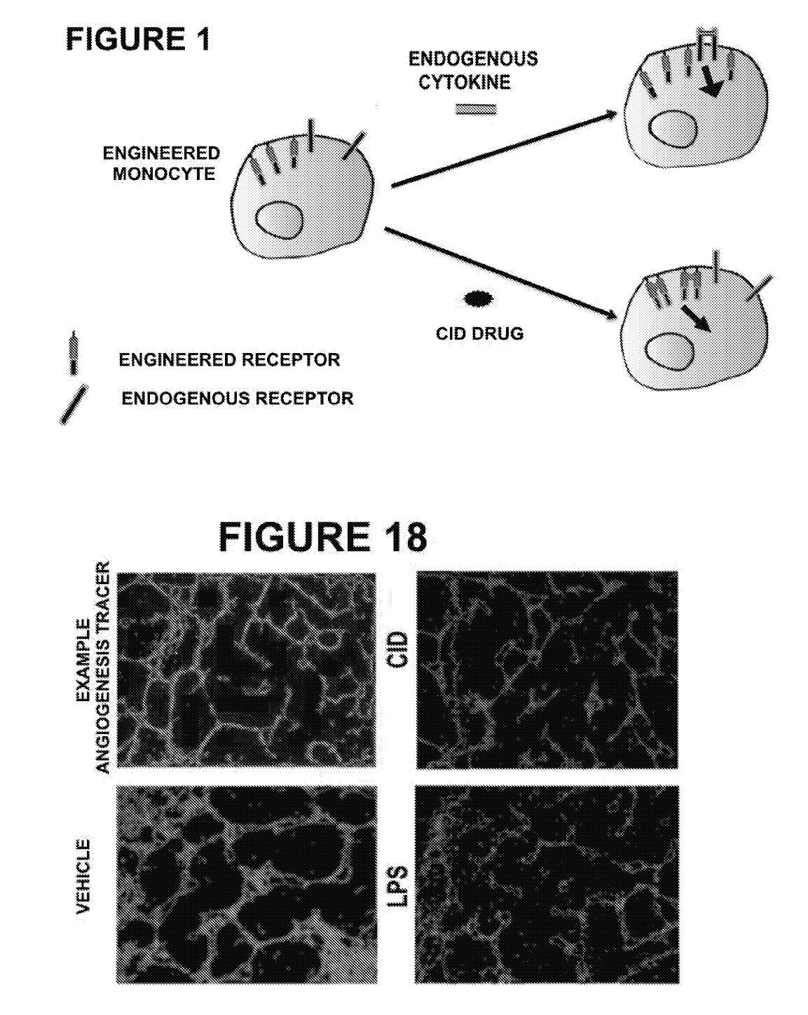

With the goal of developing inducible M1 MΦ cells, we have engineered the murine monocytic cell line RAW264.7 to express a fusion protein comprising the intracellular TLR4 signaling domain and F36V-dimerization domains that bind to a cell permeable CID drug (FIG. 1A). The cTLR4 construct is in a pCDH expression system. The 5′ end of the construct starts with a myristoylation domain (Myr), which allows targeting to the membrane to mirror the spatial localization of the endogenous full length TLR4 domain. The Myr domain is followed by the engineered F36V dimerization domain, which has a binding site for the CID drug. This domain is linked to the cTLR4 domain, which is only the cytoplasmic portion of the receptor that is necessary for proper signal transduction. This design allows dimerization via a F36V-F36V interaction with the homodimerization CID drug (AP20187). Lastly, there is a T2A ribosome skipping sequence that allows the separate expression of GFP at the 3′ end of the construct.

Delivery of the cTLR4 engineered constructs to RAW264.7 cells was achieved via lentiviral methods, Control MΦ cells were also generated. These cells were transfected with constructs lacking the cytoplasmic and engineered domain (MΦ-T2A). Confirmation that the whole engineered construct was being transcribed was validated by the expression of the GFP reporter marker in the MΦ-cTLR4 cell line. Protein expression of the cTLR4 construct in the MΦ-cTLR4 engineered cells was verified by western blot analysis for the FKBP12/F36V domain. A corresponding 35.5 kDa band can be seen in FIG. 1B.

### Pro-Inflammatory Activation of MΦ-cTLR4 Cells

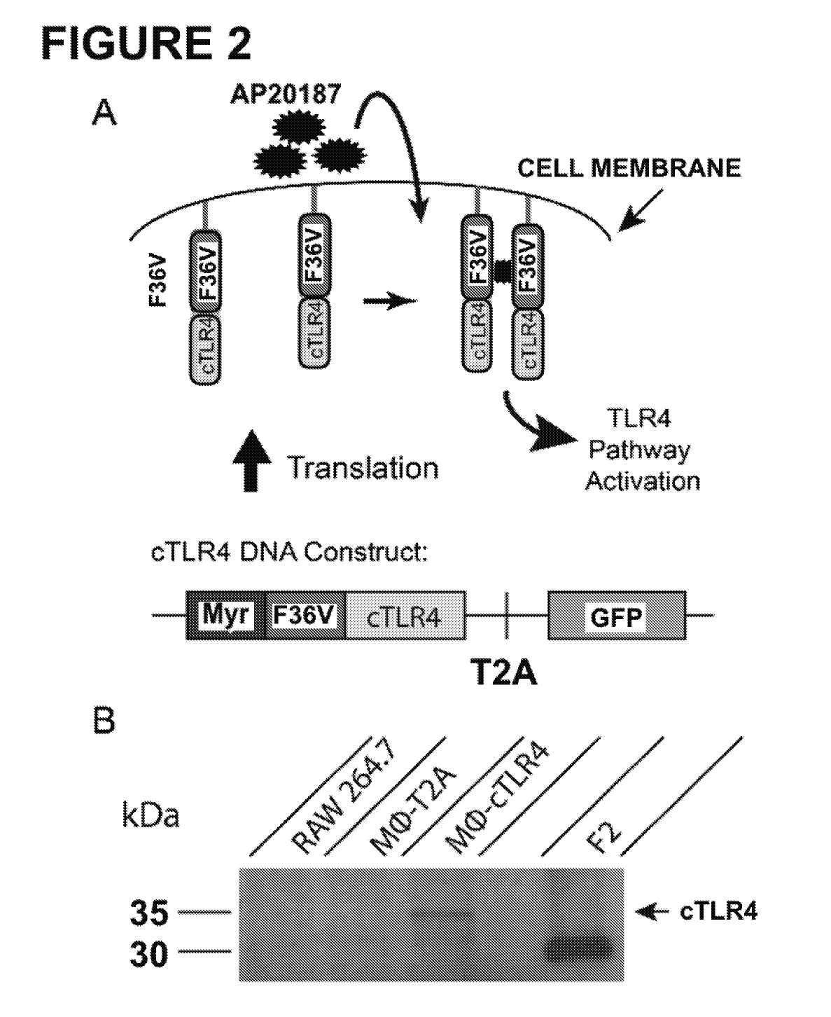

Polarized classical inflammatory MΦ are known to have increased levels of TNFα, IL-6, and iNOS.[8] Therefore the MΦ-cTLR4 cells were tested for the presence and levels of these markers. Engineered MΦ-cTLR4 cells were seeded in 6-well culture plates overnight and then treated for 24 hours with CID drug, vehicle, or LPS as a positive control. Polarization was confirmed by ELISA and western blot analyses. CID-treated MΦ-cTLR4 cells expressed increased TNFα and IL-6 levels when compared to uninduced controls (FIGS. 2A & 2B). However, the CID-treated MΦ-cTLR4 cell levels were not as high as the LPS-treated cells. We also tested for iNOS via a western blot and observed similar results with this marker. The positive control LPS-treated cells had a substantial increase in iNOS expression and there is a band evident at 130 kDa in the CID-treated lane, however, it has much lower intensity than the LPS-treated cells (FIG. 2C). The difference in LPS-treated groups and CID-treated groups can be attributed to the fact that LPS also acts on other receptors, such as CD14 and the Macrophage Scavenger Receptor, in which both of these receptor pathways can lead to NF-κB responses. Thus, there is potentially more input signal from LPS than our CID drug, which only activates through TLR4.

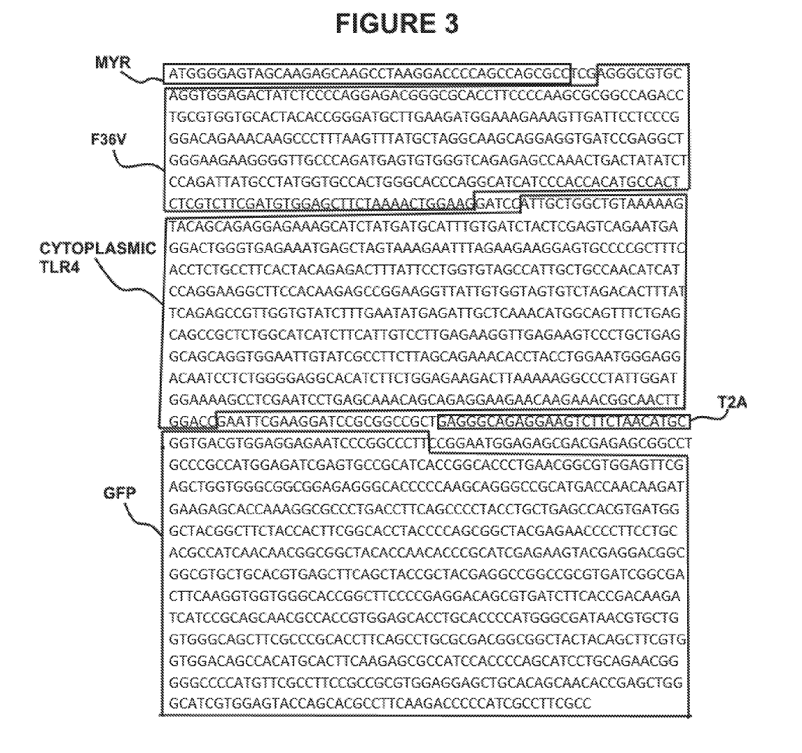

Diversity and plasticity are hallmarks of cells from the MΦ lineage and they can change phenotype depending on the surrounding microenvironment.[18] Thus, we tested how the expression of classical MΦ markers in the MΦ-cTLR4 cells were influenced by a M2 MΦ activator. Engineered cells were treated with a cocktail of either vehicle/IL-4, CID/IL-4, or LPS/IL-4, as well as the appropriate controls. The degree of polarization was assessed by ELISA and western blot analyses for IL-6, TNFα, and iNOS (FIG. 3). Both CID- and LPS-treated groups responded similarly to the IL-4 cocktail treatment and exhibited about half the expression of IL-6 and iNOS when compared to treatment groups without IL-4, However, TNFα levels for both CID drug and LPS with and without IL-4 were not significantly different.

### MΦ-cTLR4 Cells Response to CID Drug Dose, Withdrawal, and Length of Treatment

For MΦ-cTLR4 cells, IL-6 levels are elevated in CID-treated cells when compared to controls. In order to find the optimal in vitro dosage, an IL-6 ELISA was performed to test for the maximum signal of this cytokine in a CID drug titration experiment. The optimal dose of CID drug corresponds to the lowest dose that induces the highest level of IL-6 expression, The IL-6 ELISA results are seen in Supplemental FIG. 1. These results suggest that a dose of at least 50 nM, produces the maximum activation of MΦ-cTLR4 cells in the range from 50 nM-250 nM.

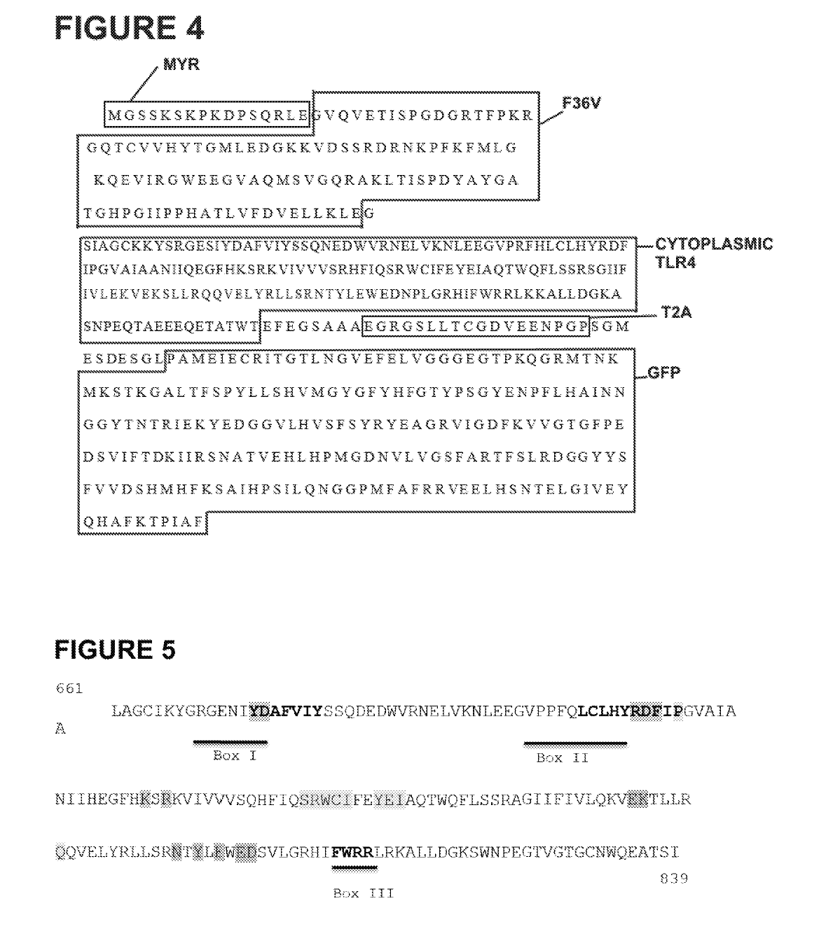

A withdrawal experiment was also performed to determine the time in which the cells would revert to a baseline state following CID drug withdrawal, MΦ-cTLR4 cells were seeded in a 6-well culture plate (1×106 cells/well). Cells were treated with vehicle, CID drug, or LPS for 24 hours. Timepoints were collected after complete CID drug withdrawal and IL-6 levels were measured at each timepoint to determine activation intensity. Results showed that cells converged to their baseline state at approximately 18 hours (FIG. 4).

In order to determine how long the engineered MΦ-cTLR4 cells would stay “on” or activated, we performed a longevity study for TNFα, IL-6, and iNOS. With constant CID drug presence in the media, we found that the MΦ-cTLR4 cells maintain considerable elevated levels of all three pro-inflammatory markers for at least 48 hours (FIG. 5A-5C). The IL-6 levels stayed activated the longest for 72 hours.

Lastly, the MΦ-cTLR4 cells were optimized for maximal signal to baseline activation by sorting four different GFP intensity populations: dim, midlow, midhigh, and high. An IL-6 ELISA was performed to determine activation of these populations compared to unsorted MΦ and MΦ-T2A populations (Supplemental FIG. 2). As signal intensity increased, the baseline activation of MΦ-cTLR4 cells also increased. A potential explanation for the high baseline activation as GFP intensity increases might be that some cells have more cTLR4 constructs integrated into their genome, thus resulting in higher GFP intensity. This higher integration will yield a greater concentration of the engineered cTLR4 construct on the cell surface and might result in self-dimerization, if the constructs are in close enough proximity. Ultimately, we determined that the midlow MΦ-cTLR4 population had similar CID and LPS activation, as well as the highest signal to noise ratio, so we used this sorted population for the remaining experiments.

### MyD88-Dependent and MyD88-Independent Signaling Pathway Activation in MΦ-cTLR4 Cells

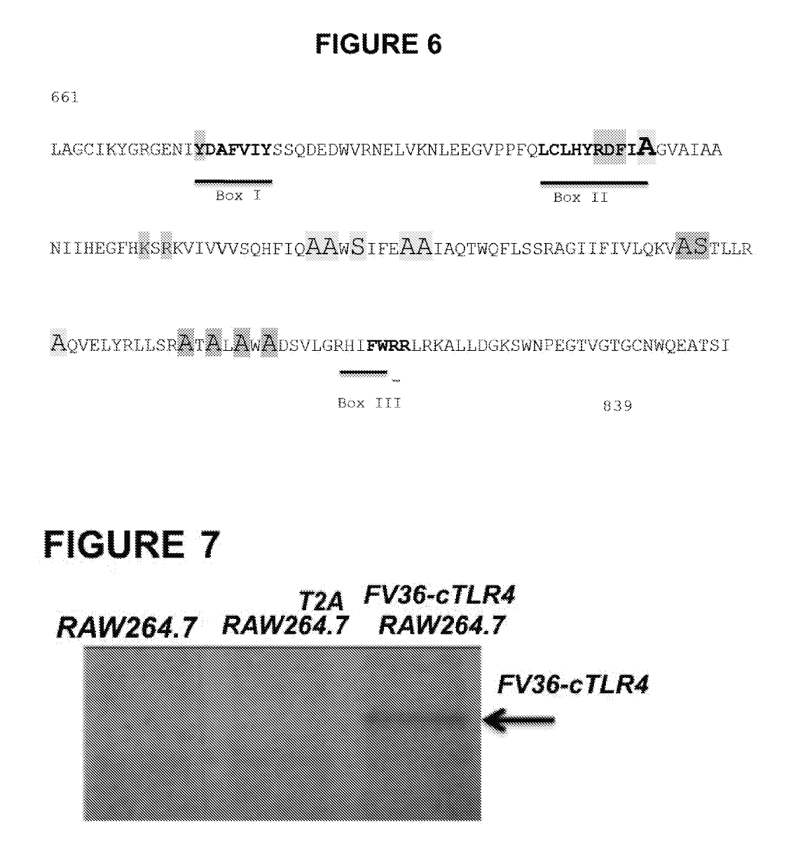

Following LPS stimulation and subsequent TNFα production, the TLR4 pathway leads to activation of NF-κB and the three MAPK pathways through the MyD88-dependent pathway. Both NF-κB and MAPK pathways directly control the transcription of the IL-6 and iNOS inflammatory genes, as well as control the mRNA stability of those transcripts. For the activated MΦ-cTLR4 cells, ERK1/2 phosphorylation is expected if the MyD88 dependent pathway and subsequent downstream TRAF6 activation has occurred. Therefore, we performed a western blot to probe for phosphorylated-ERK (p-ERK) and total ERK and compare the p-ERK/total ERK ratio relative to the zero timepoint (FIG. 6A). As time increases from 0 minutes to 60 minutes, the CID-treated MΦ-cTLR4 cells exhibit an upregulation of ERK1/2 phosphorylation at the 5 minute timepoint, a subsequent decrease for the 15 minute timepoint, and then a significant increase for the last two timepoints. The LPS-treated MΦ-cTLR4 cells exhibited a similar ERK1/2 phosphorylation pattern with lower maximum phosphorylation. The NF-κB transcription factor has also been shown to be activated following TLR4 dimerization, Thus, MΦ-cTLR4 cells were tested for NF-κB promoter activation via a Dual-Luciferase reporter assay. Cells were transduced with a NF-κB responsive promoter element driving the luciferase gene. Measurement of the luciferase activity following CID treatments (FIG. 6B) shows that CID-treated MΦ-cTLR4 cells have increased NF-κB promoter activation when compared to the vehicle. These results suggest that the CID-treated cells signal through the MyD88 dependent pathway.

To determine if the MΦ-cTLR4 cells were signaling through the MyD88-independent pathway, we tested for phosphorylated IRF3. This protein is downstream of the MyD88-independent pathway and has been shown to translocate into the nucleus and regulate type I interferon responses.[19] Western blot analysis of the p-IRF3/total IRF3 ratio relative to the zero timepoint (FIG. 7) shows a pronounced activation peak at 2 hours for both CID- and LPS-treated MΦ-cTLR4 cells when compared to vehicle. The IRF3 phosphorylation of the CID- and LPS-treated MΦ-cTLR4 cells starts to decrease following the 2 hour timepoint and subsequently reaches similar levels as vehicle at the 6 and 12 hour timepoints.

### MΦ-cTLR4 Endothelial Cell Activation

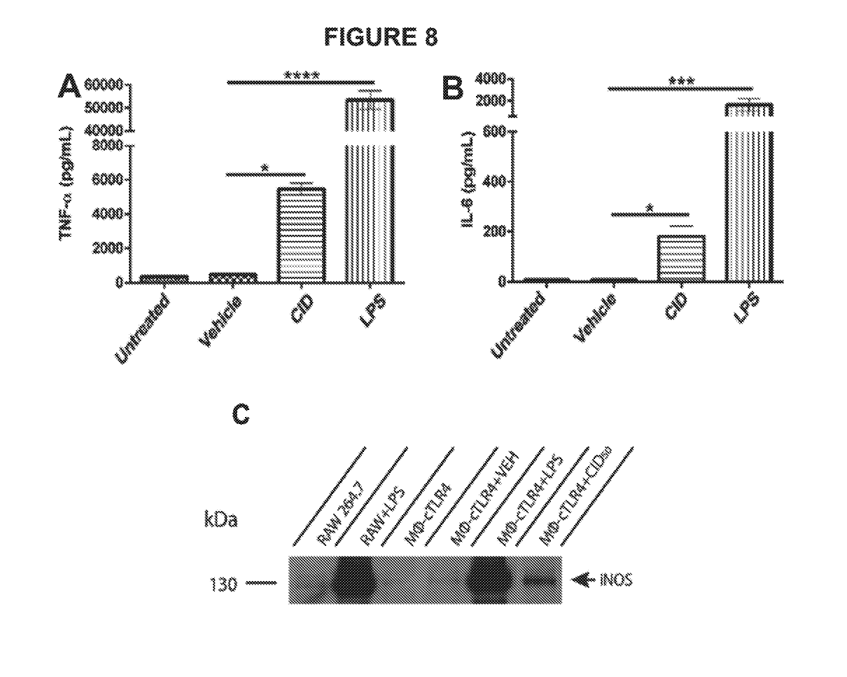

Better wound healing outcomes have been correlated with increased angiogenesis.[20] Furthermore, areas containing almost entirely pro-inflammatory MΦ have been shown to correlate with angiogenesis, in specific cases.[20, 21] Thus, we tested whether engineered MΦ-cTLR4 cells-derived factors were able to induce EC activation by measuring the expression of the VCAM-1 and ICAM-1 adhesion molecules. EC incubated with media from MΦ-cTLR4 treated with TNFα and CID drug both had increased expression of VCAM-1 and ICAM-1 (FIG. 8A & 8B) when compared to vehicle alone. For the TNFα-treatment group, 93.5% of EC were positive for VCAM-1 and 57.2% of EC were positive for ICAM-1. The CID drug-treatment group was very similar, with 93.7% of the EC being positive for VCAM-1 and 64.4% of the EC being positive for ICAM-1. The vehicle group had a baseline VCAM-1 and ICAM-1 expression when compared to the isotype control.

To determine if the TNFα was the main driver of the EC activation, we repeated the experiment with a neutralizing antibody for TNFα (FIG. 8C & 8D). Before adding the MΦ-cTLR4 conditioned media to the EC, a TNFα neutralizing antibody was incubated in the media for 15 minutes. The CID and the TNFα groups with the neutralizing antibody had severely decreased levels of both VCAM-1 and ICAM-1 when compared to the IgG control. The VCAM-1 expression was decreased by a factor of three and the ICAM-1 expression was decreased by a factor of two and fell below vehicle/baseline treatment, thus indicating that some of the activity at baseline is TNFα-dependent.

### Discussion

In this study we engineered RAW264.7 cells to have the ability to polarize into pro-inflammatory MΦ, by using the CID system for the TLR4 receptor. We confirmed that the engineered cTLR4 receptor was being expressed in the stably transduced RAW264.7 cell line. Additionally we determined that both MyD88-dependent and MyD88-independent pathways were activated by CID drug treatment in MΦ-cTLR4 cells. The CID-treated MΦ-cTLR4 cells displayed M1-like MΦ characteristics, such as increased IL-6, TNFα, and NOS expression. MΦ-cTLR4 cells were influenced by IL-4 cocktail treatment, however, the engineered cells still displayed M1 MΦ characteristics, albeit at lower expression levels. The MΦ-cTLR4 cells remained polarized in response to CID drug for at least 48 hours and CID drug withdrawal experiments suggest that the engineered cells became deactivated 18 hours after drug withdrawal. Lastly, we showed that these engineered cells have functional properties by performing a MΦ-cTLR4 conditioned-media experiment with EC. CID-polarized MΦ-cTLR4 conditioned-media had the ability to activate EC by upregulating both VCAM-1 and ICAM-1 expression on the cell surface, which are two cell adhesion molecules associated with angiogenic processes.[22, 23] Further, the activation of EC by CID-treated MΦ-cTLR4 was determined to be dependent on TNFα.

The CID system has been successfully used in literature to trigger a variety of signal transduction cascades. In vitro immunology studies using this system have been mostly focused on downstream effects of a specific engineered receptors signaling pathway.[24-26] For instance, Kuenzel et al. transfected HeLaS3 cells with a nucleotide-binding oligomerization domain-like receptor 5 (NLRCS)-FKBP fusion protein and determined that induced oligomerization of this receptor activated certain IFN signaling pathways that contributed to an antiviral defense mechanism.[25] In another study, Fooksman et al. transiently transfected T2 cell lines with a dimerizable mouse class I H2-Kb H chain-FKBP fusion protein and determined that induced dimerization, and thus clustering of this class I MHC construct, enhanced lymphoblast recognition by T cells.[26] In contrast to using the CID system to examine cause and effect relationships within a specific pathway, the present study is the first to use this system to regulate the phenotype of a cell by polarizing RAW264.7 cells into a specific pro-inflammatory MΦ. Further, our lab has previously demonstrated that this system can be used to engineer inducible bone resorbing osteoclasts from the monocyte-macrophage RAW264.7 cell line, in which it is important to note that monocytes are a common precursor to both macrophages and osteoclasts.[27]

Other groups have attempted to engineer macrophages to control the inflammatory response. For example, Wu et al, transduced MΦs in vitro with the IFN-γ gene and delivered them intratracheally to immunodeficient mice.[28] These MΦs restored immune function in the lungs of the immunodeficient mice. However, these constitutively active IFNγ-expressing pro-inflammatory MΦs probably have limited applications, since the cells were not engineered to be tunable. Additionally, Oxford BioMedica has engineered human MΦs to express cytochrome P450, which can convert a cancer prodrug into its active form during hypoxic tumor conditions. When delivered into an avascular spheroid model, the human engineered P450 MΦs were able to induce tumor cell death following the addition of the prodrug.[29] The success of this study was dependent on the hypoxia-driven expression of cytochrome P450 in MΦs. The engineered MΦ-cTLR4 in our study, on the other hand, can be controlled temporally and specifically with the addition or withdrawal of the CID drug and activation is independent of the local environment. Indeed, we observe an upregulation of key pro-inflammatory markers from these engineered MΦs as soon as 6 hours after CID drug addition and then we observe return to baseline conditions in 18 hours following drug withdrawal. The ability to tune the engineered MΦs with respect to selective activation provides a large added benefit, since the engineered MΦ-cTLR4 cells could be turned on or off when and if necessary.

Several studies have elucidated that temporal expression of key angiogenic cytokines, such as TNFα, is necessary for tip formation in EC,[30] Sainson et al. showed that 2- to 3-day pulses of TNFα in vitro and in vivo stimulates angiogenesis, as opposed to the inhibition of angiogenesis with continuous administration. We observe robust TNFα expression in our engineered pro-inflammatory MΦ-cTLR4 cells, which may possibly be utilized to promote angiogenesis, if controlled in a time-based manner. Indeed, the MΦ-cTLR4 engineered cells may be tailored to exhibit pulse behavior with the simple addition and withdrawal of CID drug at certain timepoints. In addition, we do observe that the MΦ-cTLR4-conditioned media stimulates EC activation by increasing VCAM-1 and ICAM-1 adhesion molecule expression in an in vitro setting and in a TNFα-dependent manner, which suggests that our engineered MΦ may be able to promote angiogenesis, Further, iNOS levels directly correlate with VCAM-1 expression.[31] We do see similar activation patterns with both TNFα and iNOS in our MΦ-cTLR4 cells, so both of these factors could be working in concert to upregulate adhesion molecule expression. Activation of VCAM-1 and ICAM-1 has been shown to destabilize endothelial junctions resulting in leaky vessels, a first step in the angiogenesis process. It has been suggested that a subsequent M2 MΦ phase may be necessary for the process of angiogenesis to continue and come to completion, as the M2 MΦ phenotype has been hypothesized to bridge and stabilize newly formed vessels.[32] Thus, CID drug activated MΦ-cTLR4 cells may provide the required priming step for angiogenesis to initiate.

In addition to NOS and TNFα, our CID-treated MΦ-cTLR4 cells also produce increased levels of IL-6. The IL-6 cytokine has been closely associated with promotion of angiogenesis. Increased IL-6 mRNA levels correlated with the development of ovarian follicles and the uterine lining, which are two independent physiological angiogenic processes.[33] Moreover, IL-6 treatment has been shown to promote tubule formation in brain microvessel EC in an in vitro setting. This correlated with increased IL-6 and VEGF mRNA expression in the healing adult murine brain tissue following injury.[34] These studies suggest that IL-6 may play a role in normal physiological angiogenesis as well as angiogenesis related to inflammatory remodeling of tissue. Studies in IL-6 KO mice showed that the IL-6 deletion resulted in delayed wound healing, accompanied with both delayed angiogenesis and collagen deposition.[35] The direct mechanism of IL-6 and its influence on pro-angiogenic behavior is still not completely understood, however, IL-6 seems to be a key player in this process. The present engineered MΦ-cTLR4 cells produce IL-6, along with two other factors implicated with pro-angiogenic behavior. This strongly suggests that our MΦ-cTLR4 cells may have the ability to aid in the priming of the endothelium for early stage angiogenesis.

Despite the possible use of the MΦ-cTLR4 cells as angiogenesis priming agents, in which a following M2 MΦ response might need to be necessary, these cells could also be used in certain diseases to skew the balance of a M2 MΦ-abundant process. For example, diseases characterized by excessive fibrosis could benefit from this technology, as there is often a local abundance of M2 MΦs present during fibrotic events. Fibrosis occurs due to the abundance of these M2 MΦs over-producing TGFβ, which in turn recruits fibroblasts. The recruitment of fibroblasts then leads to the overproduction of collagen, thus leading to a fibrotic state. This dysregulated process is often associated with the dense collagen fibrous capsule that surrounds an implanted material, as well as with cardiac fibrosis that plagues congestive heart failure patients.[36] A few studies have suggested that a proper balance of M1 and M2 MΦ is necessary to achieve a reduction in the extent of fibrosis.[37, 38] Thus, CID-activated MΦ-cTLR4 cells may provide a tool to reestablish the proper M1 vs M2 MΦ equilibrium and decrease the excessive collagen deposition. Another possible application of the MΦ-cTLR4 cells could be tumor inhibition. Tumor-induced angiogenesis is essential for cancer cell survival, tumor growth and metastasis propagation. An abundance of pro-angiogenic, anti-inflammatory M2 MΦs, known as tumor-associated MΦ (TAMS), is normally present in the tumor environment thus aiding tumor progression. In contrast, very few M1 MΦs able to activate NK cells and TH1 responses are present in and around the growing tumor mass[39]. Thus, the delivery of tunable MΦ-cTLR4 cells to the tumor may halt progression by activating a more pro-inflammatory immune response.

We have shown that the MΦ-cTLR4 cells become activated with the addition of CID drug and conversely that these engineered cells return to baseline conditions once CID drug has been withdrawn in an in vitro environment. However, it is still not known what will occur in vivo with the addition or withdrawal of CID drug. The IL-4 cocktail results showed that the MΦ-cTLR4 cells had decreased IL-6 and iNOS levels when compared to CID drug or LPS treatment alone, however, IL-4 did not seem to affect TNF-α levels. This suggests that the engineered cells are influenced by competing M2-like MΦ signals. These competing signals may be changing the phenotype of the engineered MΦ-cTLR4 cells into an intermediate phenotype or even skewing the cells toward a M2-like MΦ phenotype. These results are not completely surprising, as MΦ are known to be very plastic cells and can change phenotypes depending on the surrounding microenvironment. Future in vivo studies will be necessary to determine if elevated TNF-α levels, or other increased pro-inflammatory MΦ markers, are adequate enough to maintain a pro-inflammatory surrounding environment with M2 MΦ competing signals present. In a physiological setting, the CID-treated and subsequently CID-withdrawn engineered MΦs could potentially: 1) be primed to polarize into M2 pro-healing MΦs, 2) remain in a pro-inflammatory MΦ phenotype state due to the surrounding environment, 3) develop into an intermediate phenotype state due to M2 MΦ competing signals, 4) undergo apoptosis or, 5) migrate out of the inflammation site. Future studies will determine the degree of plasticity of the engineered cells, as well as how precise we can control these cells in vivo.

It has been shown previously that MΦ drive the wound healing response.[11, 40, 41] In this study, we have engineered tunable pro-inflammatory MΦ that could possibly be used to better regulate inflammation. By utilizing these MΦ-cTLR4 cells to control the host response, it might be possible to increase angiogenesis for better healing outcomes. Additionally, these engineered cells could be used as a tool to better understand and better regulate M1 MΦ-like dynamics. While RAW264.7 cells are suitable to use during in vitro inflammation studies, future investigations will focus on using a more physiological engineered primary cell type, such as bone marrow derived MΦ. [42] While ongoing studies continue to unravel MΦ-cTLR4 cell possibilities in both in vitro and in vivo settings, currently these engineered cells serve as a platform technology that could be applied to various inflammatory diseases including the FBR, fibrosis, atherosclerosis, and cancer.

### Example 2

### Plasticity of MΦ-cTLR4 Cells

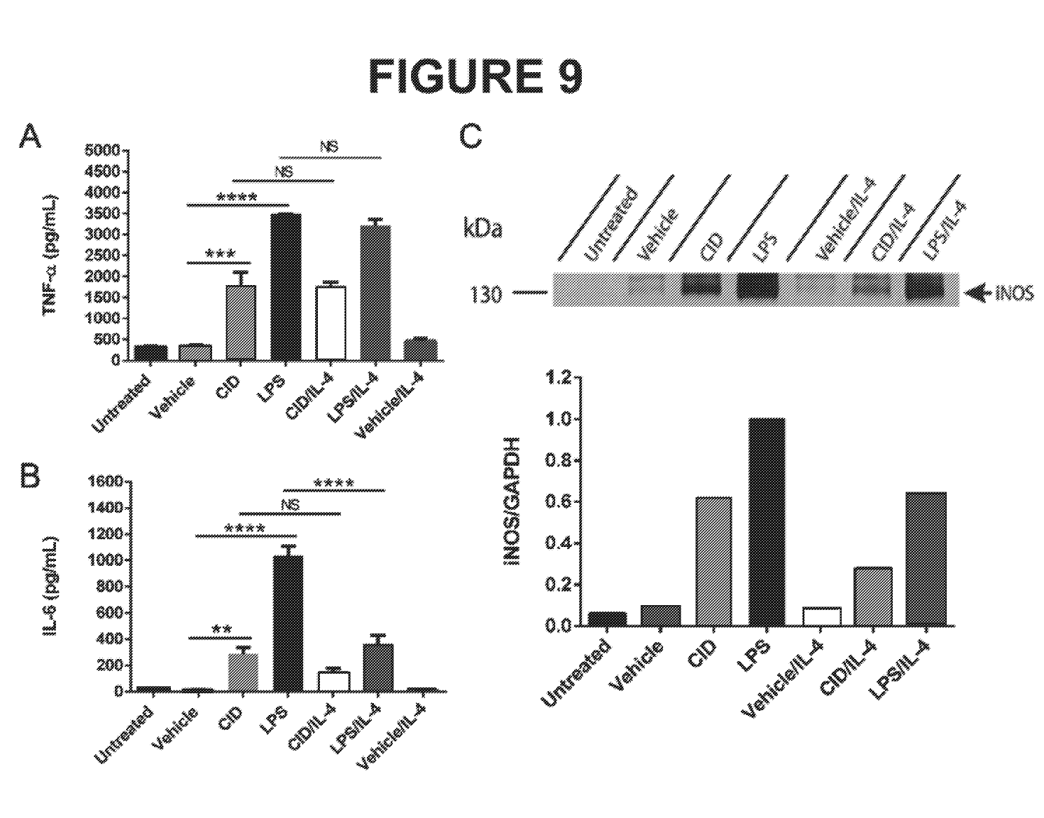

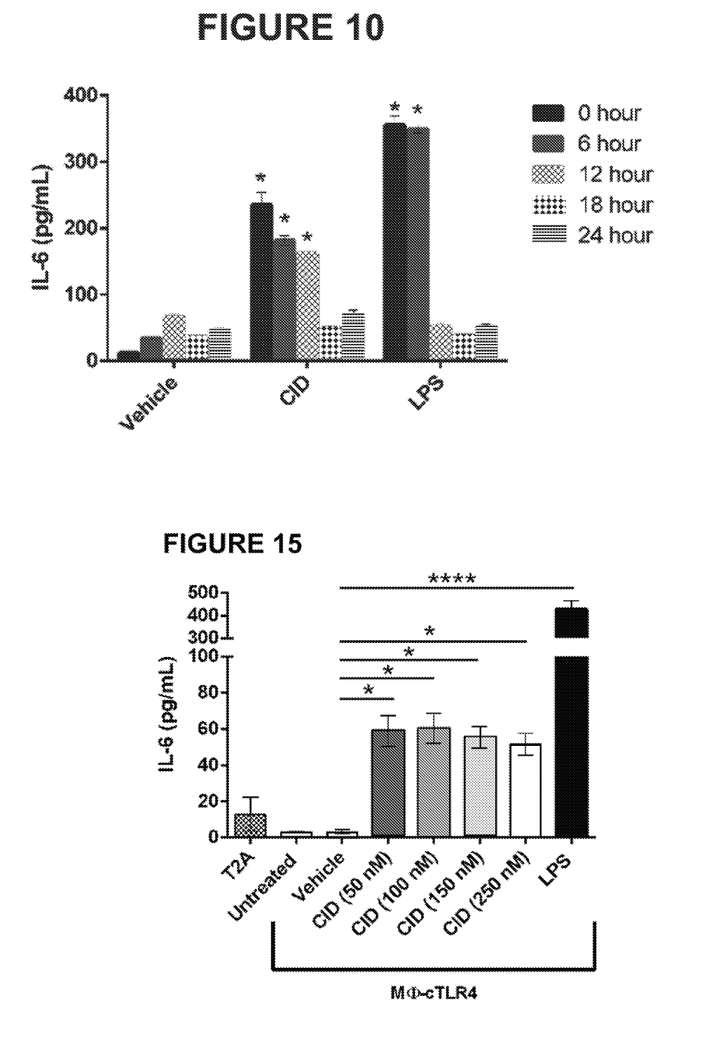

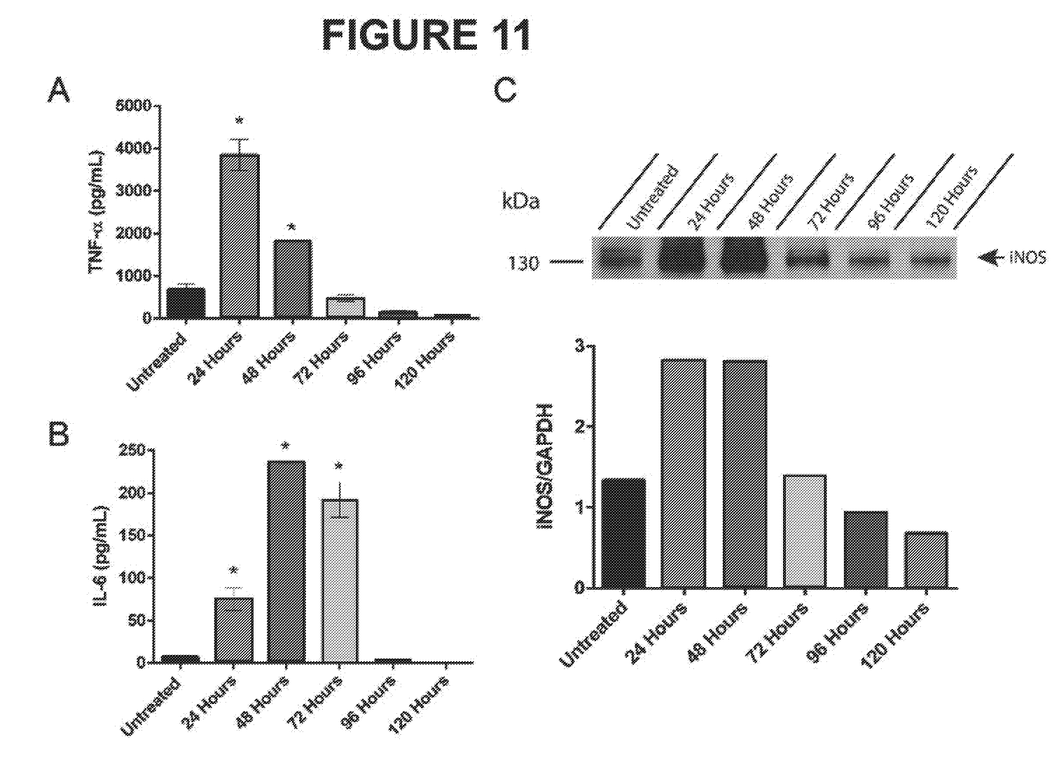

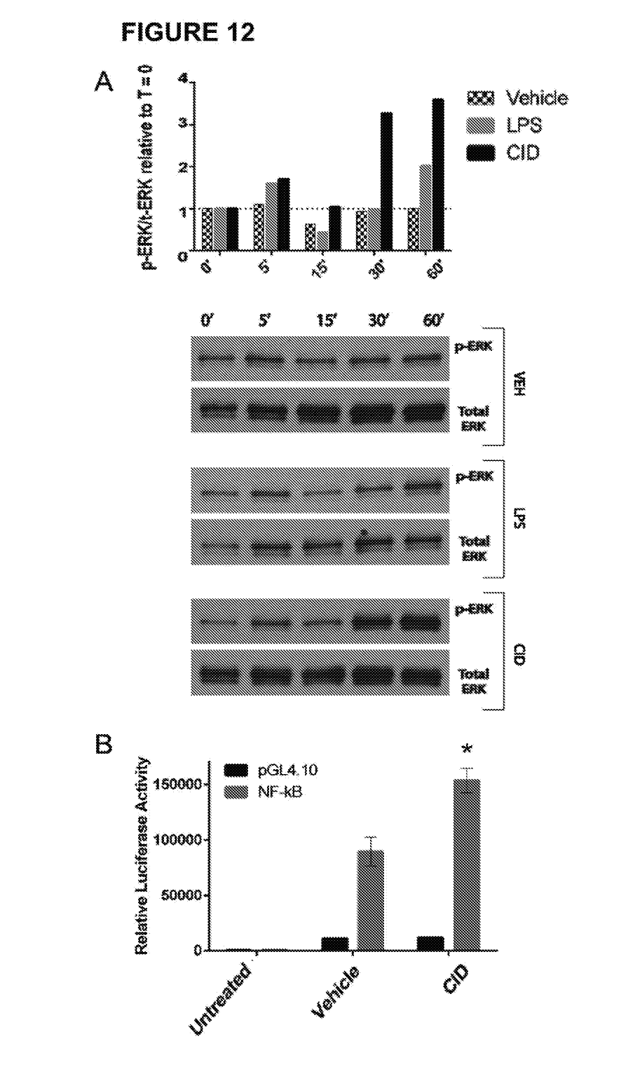

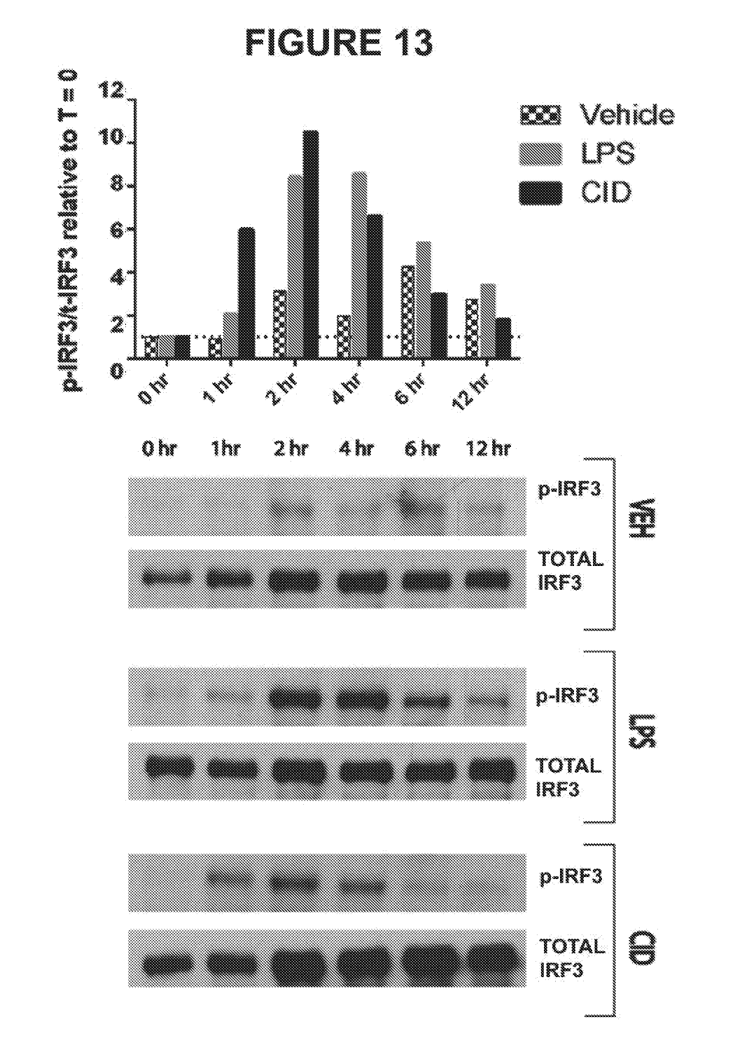

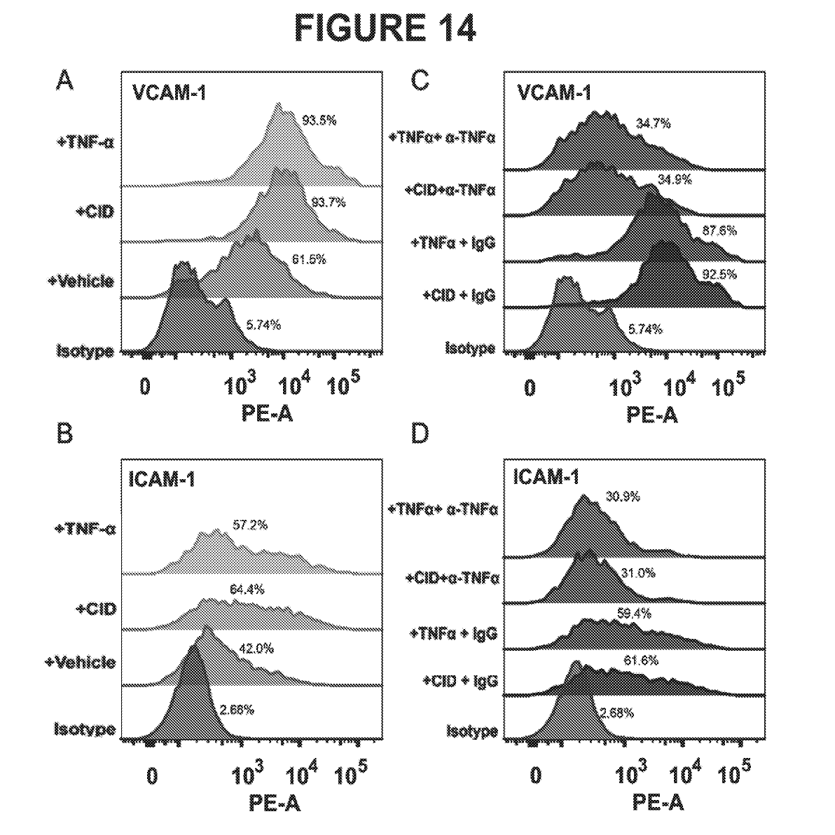

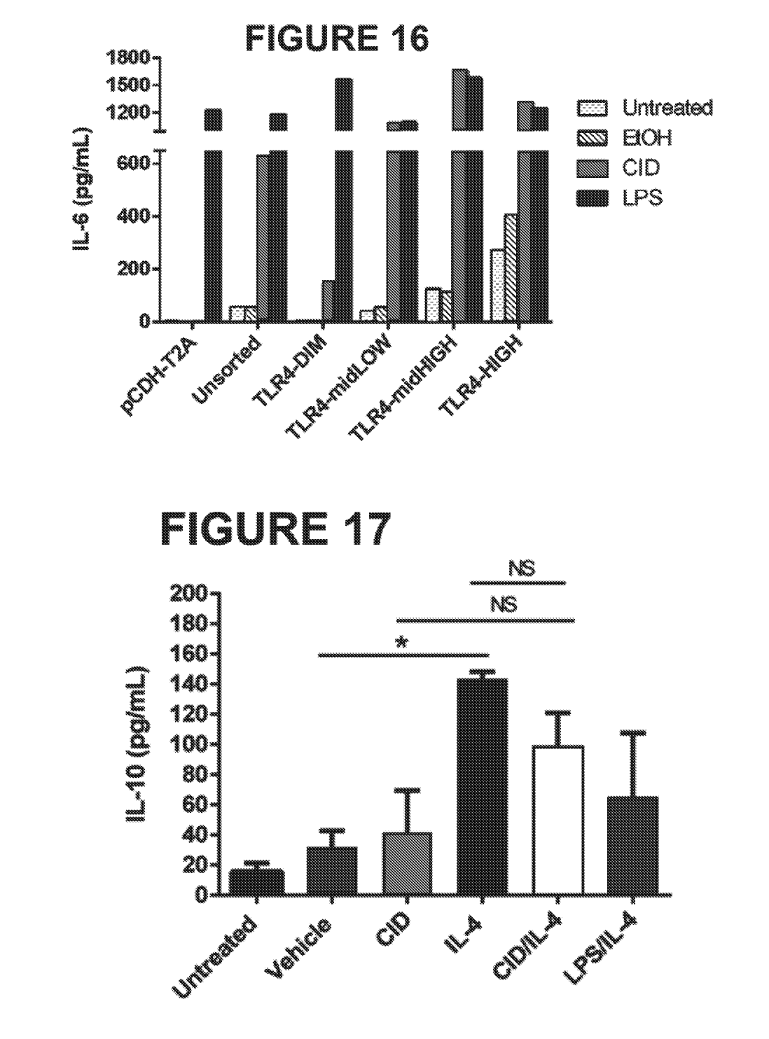

Diversity and plasticity are hallmarks of cells from the MΦ lineage and they can change phenotype depending on the surrounding microenvironment. As described in Example 1 above and in FIG. 9, we tested how the expression of classical MΦ markers in the MΦ-cTLR4 cells were influenced by a M2 MΦ activator. Engineered cells were treated with a cocktail of either vehicle/IL-4, CID/IL-4, or LPS/IL-4, as well as the appropriate controls. The degree of polarization was assessed by ELISA and western blot analyses for IL-6, TNFα, and iNOS (FIG. 9). Both CID- and LPS-treated groups responded similarly to the IL-4 cocktail treatment and exhibited about half the expression of IL-6 and iNOS when compared to treatment groups without IL-4. However, TNFα levels for both CID drug and LPS with and without IL-4 were not significantly different. Similar results to iNOS and IL-6 were observed with IL-10 cytokine expression (FIG. 17), which is a canonical pro-healing M2 MΦ marker. Cocktail treatment of CID/IL-4 and LPS/IL-4 displayed increased levels of IL-10, when compared to controls without IL-4 present.

### Example 3

### Co-Culture of MΦ-cTLR4 Cells and Endothelial Cells in Tube Formation Assay

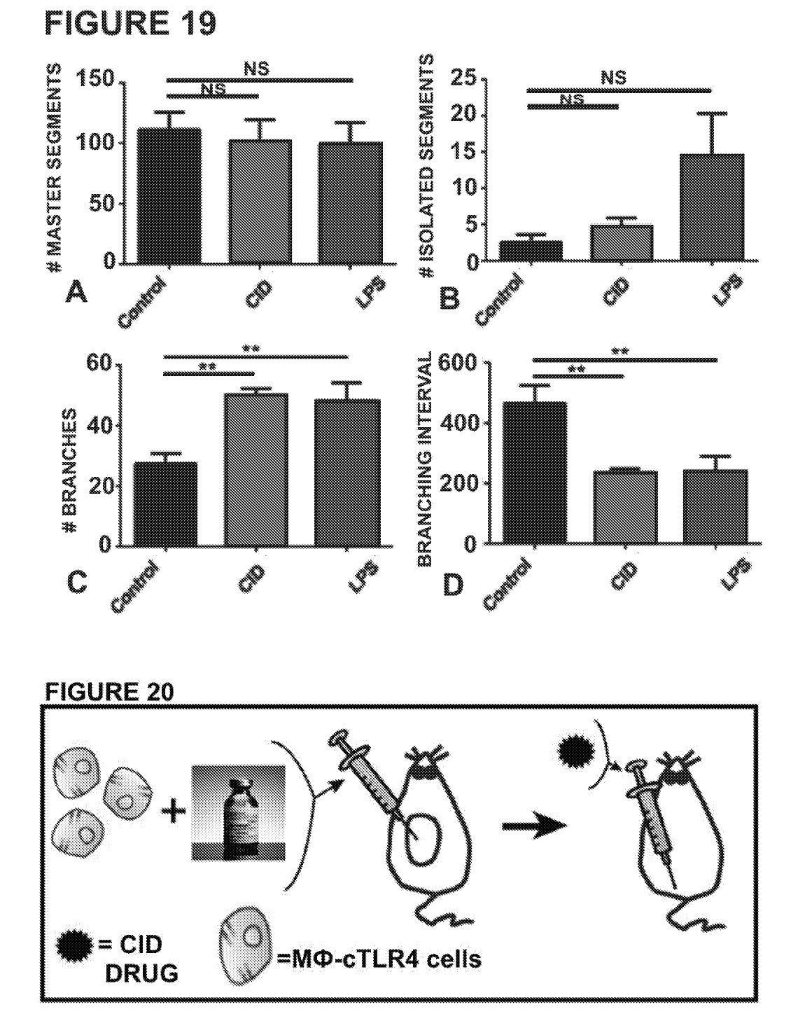

Since cell-cell interactions have been shown to be important for endothelial cells (ECs) and macrophages (MΦs), a co-culture tube formation assay was performed (FIG. 18). Untreated MΦ-cTLR4 cells co-cultured with rat aortic ECs (RAECs), with no added factors to the medium, had the least amount of isolated segments, the least amount of branches, and the most amount of branching interval. CID-treated and LPS-treated MΦ-cTLR4 cells co-cultured with RAECs, with CID or LPS added to the medium, had much more branches than untreated co-cultures with a smaller branching interval. The CID-treated group differed from the LPS-treated group in isolated segment analysis, as LPS-treated MΦc-TLR4 cells co-cultured with RAECs had the highest number of isolated segments and CID-treated MΦc-TLR4 cells co-cultured with RAECs had slightly more isolated segments than the untreated group. The number of branches and the branching interval for the CID- and LPS-treated groups were significantly different than the control. However, the number of master segments and isolated segments for the CID- and LPS-treated groups were not significantly different, when compared to the control (FIG. 19).

### Example 4

### In Vivo Analysis of MΦ-cTLR4 Cells and Their Co-Localization With iNOS Inflammation Regions in Tissue

Four groups of 4 week old female BALB/c mice (total of 16 mice) were used in this in vivo study. The four groups consisted of: 7 day non-treated mice, 7 day CID-treated mice, 14 day non-treated mice, and 14 day CID-treated mice. At day of injection, pre-plated MΦ-cTLR4 cells were lifted off and counted. 0.5×106 cells were s.c. injected mixed in with Matrigel into the right back dorsal area of the mouse. Before placing mouse back in cage, mice were injected with the first intraperitoneal CID drug dose (2 mg/kg mouse weight). Treatment groups were given CID drug injections every other day during the course of the experiment (FIG. 20).

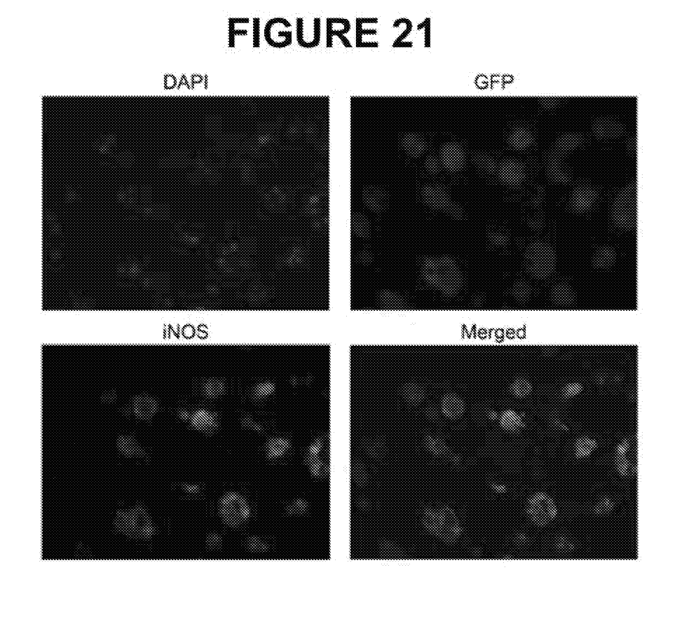

The existence of GFP positive MΦ-cTLR4 cells and iNOS co-localization indicates that the MΦ-cTLR4 cells remain functional within the Matrigel plug and also indicates the potential influence of the MΦ-cTLR4 cells on the local environment. As most cells were dead in the 14 day untreated Matrigel plugs, there were no regions of co-localization of GFP MΦ-cTLR4 cells and iNOS regions. However, the 14 day CID-treated plugs had occurrences of GFP/iNOS co-localization (FIG. 21, 14 day).

### Example 5

### Decreased Expression of VEGF in MΦ-cTLR4 Cells Treated With CID, LPS, and/or IFN-γ

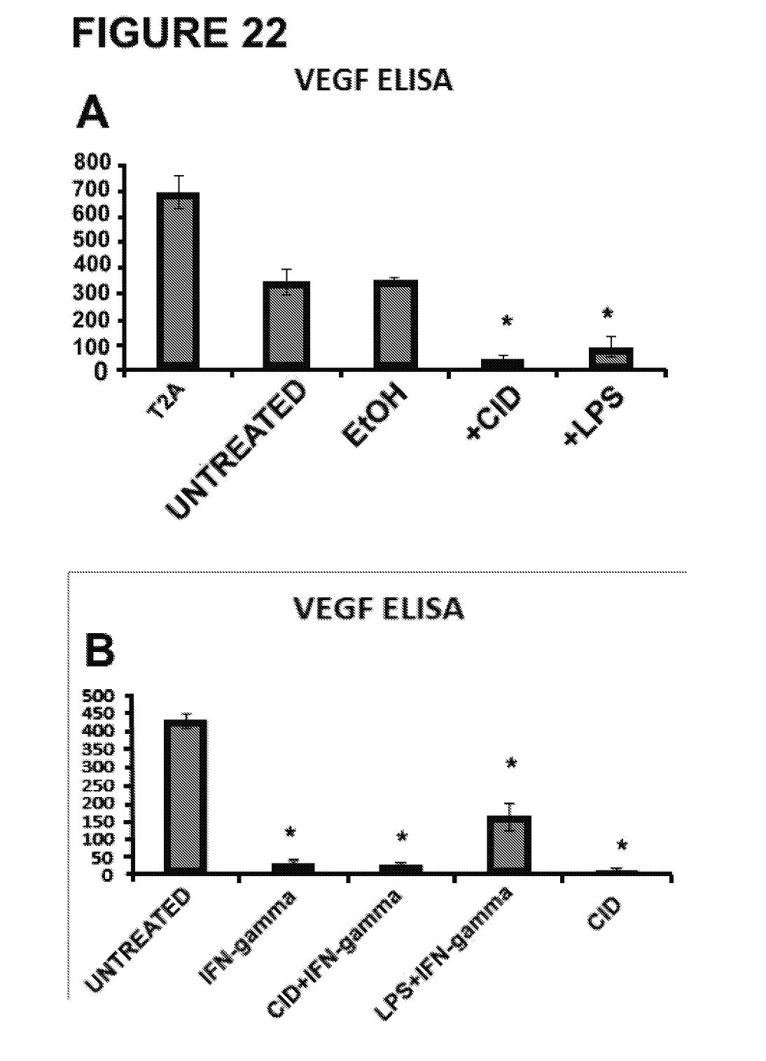

In order to determine if the MΦ-cTLR4 cells were expressing the key angiogenic molecule VEGF-A, an ELISA was performed to test for VEGF-A levels in MΦ-cTLR4 medium following various treatments (FIG. 22A). CID-treated and LPS-treated MΦ-cTLR4 cells expressed significantly decreased amounts of VEGF, when compared to controls. Since studies have shown human and rat MΦs express increased VEGF with IFN-γ treatment, the response was also tested with this factor. FIG. 22B shows cocktail treatment of IFN-γ treatment with CID or LPS, or IFN-γ alone. All groups also show a significant decrease in VEGF when compared to the control.

A significant decrease in VEGF expression was observed when MΦ-cTLR4 cells were treated with either CID, LPS, IFN-γ, or a combination of the treatments, when compared to controls. The down-regulation of VEGF could be maintaining the angiogenesis process at the first stage, which is the destabilization step. The second and third step involve sprouting and branching, respectively, which necessitates increases in VEGF expression. Since MΦ-cTLR4 cells are actively down-regulating this factor, this might play a large role in the anti-angiogenic behavior of the activated engineered cells.

### REFERENCES

1. G. L. Larsen, P. M. Henson, Annual review of immunology 1 (1983) 335-359.

2. B. M. Delavary, et al., Immunobiology 216 (2011) 753-762.

3. D. M. Mosser, J. P. Edwards, Nature Reviews Immunology 8 (2008) 958-969.

4. A. Rodriguez, et al., J. Biomedical Materials Research Part A 89 (2009) 152-159.

5. R. J. Schutte, et al., Biomaterials 30 (2009) 160-168.

6. S. Nakao, et al., The Journal of Immunology 170 (2003) 5704-5711.

7. R. J. Stewart, et al., The Journal of Immunology 156 (1996) 1221-1228.

8. A. Mantovani, et al., Trends in immunology 25 (2004) 677-686.

9. F. Mackay, et al., The Journal of experimental medicine 177 (1993) 1277-1286.

10. B. Garmy-Susini, et al., The Journal of clinical investigation 115 (2005) 1542-1551.

11. B. Behm, et al., J. European Acad. Dermatol. and Venereol. 26 (2012) 812-820.

12. F. O. Martinez, et al., Frontiers in bioscience: a journal and virtual library 13 (2008) 453.

13. C. A. Blau, et al., Proceedings of the National Academy of Sciences 94 (1997) 3076.

14. L. Jin, et al., Proceedings of the National Academy of Sciences 95 (1998) 8093.

15. L. Jin, et al., Blood 91 (1998) 890-897.

16. H. Zeng, et al., Blood 98 (2001) 328-334.

17. S. Zhao, et al., The EMBO journal 21 (2002) 2159-2167.

18. A. Sica, et al., The Journal of clinical investigation 122 (2012) 787-795.

19. A. Garcia-Sastre, et al., Science 312 (2006) 879-882.

20. S. Guo, L. A. DiPietro, Journal of dental research 89 (2010) 219-229.

21. E. M. Sussman, et al., Annals of biomedical engineering (2013) 1-9.

22. Y. Wu, et al., Circulation Research 99 (2006) 315-322.

23. A. E. Koch, et al., Angiogenesis mediated by soluble forms of E-selectin and vascular cell-adhesion molecule-1, (1995).

24. M. Sun, et al., The Journal of Immunology 177 (2006) 1481-1491.

25. S. Kuenzel, et al., The journal of immunology 184 (2010) 1990-2000.

26. D. R. Fooksman, et al., The Journal of Immunology 176 (2006) 6673-6680.

27. C. W. Rementer, et al., PloS one 8 (2013) e84465.

28. M. Wu, et al., Proc. National Academy of Sciences 98 (2001) 14589-14594.

29. L. Griffiths, et al., Gene therapy 7 (2000) 255-262.

30. R. C. Sainson, D. A. et al., Blood 111 (2008) 4997-5007.

31. F. Klug, et al., Cancer cell 24 (2013) 589-602.

32. T. Schmidt, et al., Nature 465 (2010) 697-699.

33. B. Motro, et al., Proc. National Academy of Sciences 87 (1990) 3092-3096.

34. D. Fee, et al., Cytokine 12 (2000) 655-665.

35. Z.-Q. Lin, et al., Journal of leukocyte biology 73 (2003) 713-721.

36. A. Stempien-Otero, et al., J Biol Chem 281 (2006) 15345-15351.

37. A. Meneghin, et al., Journal of Clinical Investigation 117 (2007) 530.

38. H.-J. Anders, et al., Kidney international 80 (2011) 915-925.

39, P. Allavena, et al., Critical reviews in oncology/hematology 66 (2008) 1-9.

40. T. Hunt, et al., Surgery 96 (1984) 48-54.

41. T. J. Koh, et al., Expert reviews in molecular medicine 13 (2011) e23.

42. L. M. Chamberlain, et al., Journal of Biomedical Materials Research Part A 88A (2009) 858-871.

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. (canceled)

2. A polynucleotide construct encoding a chimeric cellular receptor, the construct comprising nucleic acid sequences encoding, in operable linkage, a myristoylation sequence (Myr), a dimerizer domain, an intracellular portion of the TLR4 receptor, a ribosome skipping sequence or a cleavage sequence, and a green fluorescent protein.

3. (canceled)

4. (canceled)

5. The construct of claim 2, wherein the myristoylation sequence (Myr), the dimerizer domain, and the intracellular portion of the TLR4 receptor have the amino acid sequence of SEQ ID NO: 1.

6. The construct of claim 2, wherein the intracellular portion of the TLR4 receptor comprises an amino acid sequence having at least 90% sequence identity to the amino acid sequence of SEQ ID NO: 6, wherein SEQ ID NO: 6 is amino acid residues 661-839 of human TLR4 receptor.

7. The construct of claim 6, wherein the intracellular portion of the TLR4 receptor comprises an amino acid sequence selected from SEQ ID NOs: 7-20, or at least two mutations selected from P714A, S744A, R745A, C747S, Y751A, E752A, E775A, 776S, Q792A, N792A, Y794A, E796A, and E798A.

8. (canceled)

9. (canceled)

10. (canceled)

11. (canceled)

12. A pharmaceutical composition comprising a genetically engineered monocyte produced by a method comprising the steps of:
(a) contacting a monocyte with the construct of any claim 2 under conditions sufficient to transfect the construct into the monocyte; and
(b) culturing the monocyte transfected in step (a),
wherein the genetically engineered monocytes express the chimeric cellular receptor able to dimerize upon addition of a Chemical Inducer of Dimerization (CID) synthetic ligand, which dimerization activates signaling pathways independently of endogenous physiological ligands, thereby differentiating the transfected monocyte into a macrophage.

13. The pharmaceutical composition of claim 12, further comprising a CID synthetic ligand.

14. The pharmaceutical composition of claim 13, wherein the CID synthetic ligand is a recombinant FK506 molecule.

15. The pharmaceutical composition of claim 14, wherein the recombinant FK506 molecule is AP20187.

16. A method of reversibly inducing pro-inflammatory macrophages in a subject, the method comprising:
(a) administering the composition of claim 12 to a subject; and
(b) administering a CID synthetic ligand to the subject;
whereby the CID synthetic ligand activates the genetically engineered monocytes.

17. The method of claim 16, further comprising:
(c) withdrawing administration of the CID synthetic ligand and/or administering a washout ligand, thereby reversing the activation of the genetically engineered monocytes.

18. The method of claim 16, wherein the administering of step (a) is by implantation into a target organ, injection into a target tissue, introduction of a scaffold to a target site, or intravenous administration.

19. The method of claim 16, wherein the genetically engineered monocyte is pre-treated with a CID synthetic ligand prior to the administering of step (a).

20. The method of claim 16, wherein the composition is administered to treat a fibrotic disease.

21. The method of claim 20, wherein the fibrotic disease is selected from the group consisting of pulmonary fibrosis, cardiac fibrosis, and foreign body reaction.

22. The method of claim 16, wherein the composition is administered to treat an inflammatory disease.

23. The method of claim 22, wherein the inflammatory disease is a chronic inflammatory disease.

24. The method of claim 23, wherein the chronic inflammatory disease is selected from the group consisting of atherosclerosis and rheumatoid arthritis,

25. The method of claim 16, wherein the composition is administered to treat cancer inflammation.
